(19) United States
(12) Patent Application Publication
(10) Pub. No.: US 2025/0215491 A1
(43) Pub. Date: Jul. 3, 2025

(54) SEQUENCING WITH SINGLE-STRANDED BINDING PROTEINS

(71) Applicant: ILLUMINA, INC., San Diego, CA (US)

(72) Inventors: Christopher Winnard, Cambridge (GB); Amy Charles, Cambridge (GB); Philip Balding, Cambridge (GB); Justin Robbins, San Diego, CA (US)

(73) Assignee: ILLUMINA, INC., San Diego, CA (US)

(21) Appl. No.: 18/982,235

(22) Filed: Dec. 16, 2024

Related U.S. Application Data
(60) Provisional application No. 63/615,368, filed on Dec. 28, 2023.

(51) Int. Cl. C12Q 1/6869

(52) U.S. Cl. CPC C12Q 1/6869

(57) ABSTRACT

Methods and compositions for polynucleotide sequencing. The methods include incorporating a blocked, labeled nucleotide into a copy polynucleotide strand; detecting the identity of the blocked, labeled nucleotide; and removing the blocked, labelled nucleotide. The steps may be repeated. At least one of the steps is performed in the presence of a single-stranded binding protein.

### RELATED APPLICATION

This application claims the benefit of U.S. Provisional Application No. 63/615,368 filed on Dec. 28, 2023, which is hereby incorporated herein by reference in its entirety to the extent that it does not conflict with the disclosure presented herein.

### SEQUENCE LISTING

The instant application contains a Sequence Listing which has been submitted electronically in XML format and is hereby incorporated by reference in its entirety. Said XML copy, created on Dec. 16, 2024, is named 531.002723-ST26 and is 16 kilobytes in size.

### FIELD

The present disclosure relates to, among other things, single-stranded sequencing of polynucleotides.

### INTRODUCTION

Many sequencing methods, such as sequencing by synthesis methods, use a single-stranded oligonucleotide as a template. Typically, such single-stranded oligonucleotides may be rapidly and accurately sequenced. However, single-stranded oligonucleotides having sequences that may self-hybridize to form secondary structures present unique oligonucleotide sequencing challenges. For example, single-stranded oligonucleotides that form secondary structures such as G-quadruplexes, stem loops, hairpins, or other self-hybridizing structures are difficult to sequence. The formation of the secondary structure in a single-stranded oligonucleotide can result in sequencing errors and/or the inability to sequence that portion of the polynucleotide.

Sequencing of a template polynucleotide strand may occur through multiple cycles of steps by which one detectable nucleotide per cycle is incorporated into a copy strand complementary to the template strand. The detectable nucleotides are typically blocked to prevent incorporation of more than one detectable nucleotide per cycle. After an incubation time, a wash step is typically performed to remove any unincorporated detectable nucleotide. A detection step, in which the identity of the detectable nucleotide incorporated into the copy strand is determined, may then performed. Next, an unblocking step and cleavage or masking step is performed in which the blocking agent is removed from the last incorporated nucleotide in the copy strand, and the detectable moiety is cleaved from or masked on the last nucleotide incorporated into the copy strand. In some instances, the step of removing the blocking moiety also removes the detectable moiety. The cycle is then repeated by introducing blocked, detectable nucleotides in an incorporation step. At any step during the sequencing of a template polynucleotide, a portion of the single-stranded template polynucleotide may form a secondary structure preventing sequencing or resulting in sequencing errors.

As such, it would be desirable to develop sequencing methodologies that allow for the accurate and robust sequencing of oligonucleotides that are capable of forming secondary structures.

### SUMMARY

The present disclosure describes, among other things, polynucleotide sequencing methods that employ a single-stranded binding protein in one or more of the sequencing steps. Inclusion of the single-stranded binding protein in one or more of the sequencing steps may prevent the formation of a single-stranded polynucleotide template from forming a secondary structure.

The present disclosure describes a polynucleotide sequencing method. The method includes: (a) incorporating a blocked, labeled nucleotide into a copy polynucleotide strand that is complementary to and hybridized with at least a portion of a template polynucleotide strand in a sequencing complex; (b) detecting the identity of the blocked, labeled nucleotide; (c) chemically removing a label and blocking moiety from the blocked, labeled nucleotide incorporated into the copy strand; and (d) repeating steps (a)-(c). At least one or the steps (a)-(c) are performed int the presence of a single-stranded binding protein.

In some embodiments, the method is a method for improving secondary structure resolution of a polynucleotide sequencing by synthesis process. In some such embodiments, the method is for improving G-quadruplex resolution. In some embodiments, the method includes repeating steps (a)-(c) to sequence at least a portion of a plurality of polynucleotide templates having the same sequence. Completion of sequencing the at least a portion of a plurality of polynucleotides results in a resolution value (a percent of nucleotides correctly identified in a one or more regions of known secondary structure in the sequenced plurality of polynucleotide templates) that is larger than a resolution value of the same method completed without a single-stranded binding protein.

In some embodiments, the method is a method for lowering the error rate of a polynucleotide sequencing by synthesis process. In some embodiments, the method includes repeating steps (a)-(c) at least 50 times to sequence at least a portion of a plurality of polynucleotide templates having the same sequence. Completion of sequencing the at least a portion of a plurality of polynucleotides results in an error rate (a percent of nucleotides incorrectly identified in the sequenced plurality of polynucleotide templates) that is lower than an error rate of the same method completed without a single-stranded binding protein.

In some embodiments, increasing incorporation kinetics of a polynucleotide sequencing by synthesis process. In some such embodiments, step (a) is performed in the presence of a single-stranded binding protein. In some embodiments, completion of step (a) in the presence of a single-stranded binding protein results in an incorporation rate, and wherein the incorporation rate is faster than an incorporation rate of performing step (a) without the single-stranded binding protein.

In some embodiments, step (a) further comprises exposing the sequencing complex to an incorporation composition; step (b) further comprises exposing the sequencing complex to a detection composition; step (c) further comprises exposing the sequencing complex to a cleavage composition; and wherein at least one of the incorporation composition, the detection composition, and cleavage composition comprise the single-stranded binding protein.

The details of one or more embodiments are set forth in the accompanying drawings and the description below. Other features, objects, and advantages will be apparent from the description and drawings, and from the claims.

It is to be understood that both the foregoing general description and the following detailed description present embodiments of the subject matter of the present disclosure and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### DESCRIPTION OF DRAWINGS

The following detailed description of specific embodiments of the present disclosure may be best understood when read in conjunction with the following drawings.

FIG. 1 is a flow diagram illustrating an overview of sequencing method consistent with some embodiments of the present disclosure.

FIG. 2 is a schematic top plan view of a cartridge including compositions for sequencing in accordance with various embodiments disclosed herein.

FIG. 3 is a schematic plan view of an embodiment of a flow cell that may be employed in accordance with the teachings presented herein.

FIG. 4 is a plot of percent error rate per cycle for polynucleotide templates sequenced by synthesis where the incorporation composition and wash composition included a single-stranded binding protein.

FIG. 5 are plots of G-quadruplex callability and G-quadruplex coverage for polynucleotide templates sequenced by synthesis where the incorporation composition and wash composition or the detection composition included a single-stranded binding protein.

FIG. 6 is a plot of percent bases soft-clipped for polynucleotide templates sequenced by synthesis where the incorporation composition included or was free of various components.

FIG. 7 is a plot showing the percent error, P90 blue, and P90 green for polynucleotide templates sequenced with and without a single-stranded binding protein being present in the detection step.

FIG. 8 is a plot showing the percent improvement in incorporation rate for various incorporation conditions.

FIG. 9 are plots showing mean phasing and mean prephasing, Q30, and percent error rate for polynucleotide templates sequenced by synthesis where the incorporation step was performed in the presence of various single-stranded binding proteins.

FIG. 10 is a plot showing the G-quadruplex resolution value for polynucleotide templates sequenced by synthesis where a single-stranded binding protein was present during the incorporation step using two polymerase conditions.

FIG. 11 is a plot showing the mean number of difficult G-quads for polynucleotide templates sequenced by synthesis where a single-stranded binding protein was present during the incorporation step or present during the detection step.

FIG. 12 shows binding isotherms for Thermus thermophilus (Tth) single stranded-stranded binding protein (Tth SSB) and Tth F225P single-stranded binding protein binding (Tth SSB F255P) to single-stranded DNA.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### Definitions

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. Thus, for example, reference to a “template polynucleotide sequence” includes examples having two or more such “template polynucleotide sequences” unless the context clearly indicates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

As used herein, “have”, “has”, “having”, “include”, “includes”, “including”, “comprise”, “comprises”, “comprising” or the like are used in their open-ended inclusive sense, and generally mean “include, but not limited to”, “includes, but not limited to”, or “including, but not limited to”.

“Optional” or “optionally” means that the subsequently described event, circumstance, or component, can or cannot occur, and that the description includes instances where the event, circumstance, or component, occurs and instances where it does not.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

In addition, the recitations herein of numerical ranges by endpoints include all numbers subsumed within that range (e.g., 1 to 5 includes 1, 1.5, 2, 2.75, 3, 3.80, 4, 5, etc.). Where a range of values is “greater than”, “less than”, etc. a particular value, that value is included within the range.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. However, it will be understood that a presented order is one embodiment of an order by which the method may carried out. Any recited single or multiple feature or aspect in any one claim may be combined or permuted with any other recited feature or aspect in any other claim or claims.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising an incorporation step, a detection step, a deprotection step, and one or more wash steps includes embodiments where the method consists of enumerated steps and embodiments where the method consists essentially of the enumerated.

As used herein, “providing” in the context of a compound, composition, or article means making the compound, composition, or article, purchasing the compound, composition or article, or otherwise obtaining the compound, composition or article.

As used herein, the term “chain extending enzyme” is an enzyme that produces a copy replicate of a polynucleotide using the polynucleotide as a template strand. For example, the chain extending enzyme may be an enzyme having polymerase activity. Typically, DNA polymerases bind to the template strand and then move down the template strand sequentially adding nucleotides to the free hydroxyl group at 3′ end of a growing strand of nucleic acid. DNA polymerases typically synthesize complementary DNA molecules from DNA templates and RNA polymerases typically synthesize RNA molecules from DNA templates (transcription). Polymerases may use a short RNA or DNA strand, called a primer, to begin strand growth. Some polymerases may displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity. Any suitable polymerase may be used with the methods and/or compositions (e.g., kits) of the present disclosure. In some embodiments, the polymerase is a polymerase described in U.S. application Ser. No. 18/373,620 filed Sep. 27, 2023, US Patent Application Number U.S. Ser. No. 16/703,569 (U.S. Pat. No. 11,001,816B2), PCT Application Number PCT/US2013/03169 (WO2014142921A1) all of which are hereby incorporated by reference in its entirety.

As used herein, the term “primer” and its derivatives refer generally to any polynucleotide that may hybridize to a target sequence of interest. Typically, the primer functions as a substrate onto which nucleotides may be polymerized by a polymerase; in some embodiments, however, the primer may become incorporated into the synthesized polynucleotide strand and provide a site to which another primer may hybridize to prime synthesis of a new strand that is complementary to the synthesized nucleic acid molecule. The primer may be comprised of any combination of nucleotides or analogs thereof. In some embodiments, the primer is a single-stranded oligonucleotide or polynucleotide.

The terms “polynucleotide,” “oligonucleotide,” and “oligonucleotide strand” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). As used herein, “amplified target sequences” and its derivatives, refers generally to a polynucleotide sequence produced by the amplifying the target sequences using target-specific primers and the methods provided herein. The amplified target sequences may be either of the same sense (i.e the positive strand) or antisense (i.e., the negative strand) with respect to the target sequences.

The term “polynucleotide template” or “template polynucleotide” refer to a polymeric form of a nucleotide that includes a target nucleic acid and an adaptor on one or both ends.

Suitable nucleotides for use in the provided methods include, but are not limited to, deoxynucleotide triphosphates, deoxyadenosine triphosphate (dATP), deoxythymidine triphosphate (dTTP), deoxycytidine triphosphate (dCTP), and deoxyguanosine triphosphate (dGTP). Optionally, the nucleotides used in the provided methods, whether labeled or unlabeled, can include a blocking moiety such as a reversible terminator moiety that inhibits chain extension. Suitable labels for use on the labeled nucleotides include, but are not limited to, haptens, radionucleotides, enzymes, fluorescent labels, chemiluminescent labels, and chromogenic agents.

A polynucleotide will generally contain phosphodiester bonds, although in some cases nucleic acid analogs can have alternate backbones, comprising, for example, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Polynucleotides containing one or more carbocyclic sugars are also included within the definition of polynucleotides (see Jenkins et al., Chem. Soc. Rev. (1995) pp169-176). Several polynucleotide analogs are described in Rawls, C & E News Jun. 2, 1997 page 35. All these references are hereby expressly incorporated by reference. These modifications of the ribose-phosphate backbone may be done to facilitate the addition of labels, or to increase the stability and half-life of such molecules in physiological environments.

A polynucleotide will generally contain a specific sequence of four nucleotide bases: adenine (A); cytosine (C); guanine (G); and thymine (T). Uracil (U) can also be present, for example, as a natural replacement for thymine when the nucleic acid is RNA. Uracil can also be used in DNA. A polynucleotide may also include native or non-native bases. In this regard, a native deoxyribonucleic acid polynucleotide may have one or more bases selected from the group consisting of adenine, thymine, cytosine or guanine and a ribonucleic acid may have one or more bases selected from the group consisting of uracil, adenine, cytosine or guanine. It will be understood that a deoxyribonucleic acid polynucleotide used in the methods or compositions set forth herein may include, for example, uracil bases and a ribonucleic acid can include, for example, a thymine base. Exemplary non-native bases that may be included in a nucleic acid, whether having a native backbone or analog structure, include, without limitation, inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. Optionally, isocytosine and isoguanine may be included in a nucleic acid in order to reduce non-specific hybridization, as generally described in U.S. Pat. No. 5,681,702, which is incorporated by reference herein in its entirety.

A non-native base used in a polynucleotide may have universal base pairing activity such that it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine, which basepairs with cytosine, adenine or uracil.

Incorporation of a nucleotide into a polynucleotide strand refers to joining of the nucleotide to a free 3′ hydroxyl group of the polynucleotide strand via formation of a phosphodiester linkage with the 5′ phosphate group of the nucleotide. The polynucleotide template to be sequenced can be DNA or RNA, or even a hybrid molecule that includes both deoxynucleotides and ribonucleotides. The polynucleotide can include naturally occurring and/or non-naturally occurring nucleotides and natural or non-natural backbone linkages.

The terms “primer oligonucleotide”, “oligonucleotide primer”, and “primer” are used throughout interchangeably and are polynucleotide sequences that are capable of annealing specifically to one or more polynucleotide templates to be amplified or sequenced. Generally, primer oligonucleotides are single-stranded or partially single-stranded. Primers may also contain a mixture of non-natural bases, non-nucleotide chemical modifications or non-natural backbone linkages so long as the non-natural entities do not interfere with the function of the primer. Typically, the primer functions as a substrate onto which nucleotides may be polymerized by a polymerase; in some embodiments, however, the primer may become incorporated into the synthesized polynucleotide strand and provide a site to which another primer may hybridize to prime synthesis of a new strand that is complementary to the synthesized nucleic acid molecule. The primer may include any combination of nucleotides or analogs thereof. In some embodiments, the primer is a single-stranded oligonucleotide or polynucleotide.

As used herein, the term “adapter” and its derivatives, e.g., universal adapter, refers generally to any linear oligonucleotide which can be ligated to a target nucleic acid. In some embodiments, the adapter is substantially non-complementary to the 3′ end or 5′ end of any target sequence present in a sample. In some embodiments, suitable adapter lengths are in the range of about 10-100 nucleotides, about 12-60 nucleotides and about 15-50 nucleotides in length. Generally, the adapter can include any combination of nucleotides and/or nucleic acids. In some embodiments, the adapter can include one or more cleavable groups at one or more locations. In some embodiments, the adapter can include a sequence that is substantially identical, or substantially complementary, to at least a portion of a primer. In some embodiments, the adapter can include a sequence that is substantially identical, or substantially complementary, to at least a portion of a surface oligonucleotide. In some embodiments, the adapter can include a barcode, also referred to as an index or tag, to assist with downstream error correction, identification, or sequencing. The terms “adaptor” and “adapter” are used interchangeably.

As used herein, the term “universal sequence” refers to a region of sequence that is common to two or more target nucleic acids, where the molecules also have regions of sequence that differ from each other. A universal sequence that is present in different members of a collection of molecules can allow capture of multiple different nucleic acids using a population of capture nucleic acids that are complementary to a portion of the universal sequence, e.g., a universal capture binding sequence. Non-limiting examples of universal capture binding sequences include sequences that are identical to or complementary to P5 and P7 primers. Similarly, a universal sequence present in different members of a collection of molecules can allow the replication or amplification of multiple different nucleic acids using a population of universal primers that are complementary to a portion of the universal sequence, e.g., a universal primer binding site. Target nucleic acid molecules may be modified to attach universal adapters (also referred to herein as adapters), for example, at one or both ends of the different target sequences, as described herein.

As used herein, the term “nucleic acid” is intended to be consistent with its use in the art and includes naturally occurring nucleic acids and functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from adenine, thymine, cytosine or guanine and a ribonucleic acid can have one or more bases selected from uracil, adenine, cytosine or guanine. Useful non-native bases that can be included in a nucleic acid are known in the art. The term “target,” when used in reference to a nucleic acid (e.g., “nucleic acid target” or “target nucleic acid”) is intended as a semantic identifier for the nucleic acid in the context of a method or composition set forth herein and does not necessarily limit the structure or function of the nucleic acid beyond what is otherwise explicitly indicated. A “target nucleic acid” having an adapter at one or more ends, is referred to as a polynucleotide template.

### DETAILED DESCRIPTION

Presented herein are methods and compositions relating to sequencing polynucleotides. Specifically, the present disclosure provides sequencing methods, compositions, kits, and cartridges that employ or contain a single-stranded binding protein. The inclusion of a single-stranded binding protein during one or more steps of polynucleotide sequencing may prevent the template polynucleotide strand from self-hybridizing and forming secondary structures (e.g., G-quadruplexes) that present challenges to sequencing. For example, secondary structures present in a template polynucleotide during sequencing may result in increased sequencing errors.

### Polynucleotide Template Strand Secondary Structures

The methods, compositions, kits, and cartridges of the present disclosure may be used to sequence any polynucleotide template strand. The methods, compositions, kits, and cartridges of the present disclosure may be particularly useful for sequencing polynucleotide template strands that have an increased likelihood of self-hybridization throughout the sequencing process. Self-hybridization of a template polynucleotide strand may result in one or more secondary structures. Examples of polynucleotide secondary structures include G-quadruplexes (G-quad or G4), stem-loops, and hairpins. For example, in some embodiments, the methods, compositions, kits, and cartridges may be employed when a polynucleotide template strand is predicted, or is known, to have regions of self-complementarity and/or guanine rich regions.

In some embodiments, the methods, compositions, kits, and cartridges of the present disclosure may be used to reduce the likelihood of G-quad formation in a template polynucleotide strand. G-quads are secondary structures formed by the association of two or more G-quartets by pi-pi stacking. A G-quartet is a quasi-co-planar structure including a hydrogen bonding network between four guanines. A G-tract is a portion of a nucleotide sequence that includes two consecutive guanines. Two or more G-tracts can be linked on the same polynucleotide strand through loops (propeller loops, V-shaped loops, lateral loops, diagonal loops, snapback loops, and the like), bulges, or other sequences to from a G-quartet. G-quadruplexes may have a variety of conformations, include various numbers of G-quartets, have various G-tract strand orientations, and include other features.

The likelihood of a polynucleotide template strand of forming one or more secondary structures depends at least in part on the sequence of the polynucleotide template strand. For example, a template polynucleotide strand having regions of self-complementarity may be more likely to self-hybridize. Additionally, a template polynucleotide strand having a high guanine content may be more likely to form one or more G-quads.

### Single-Stranded Binding Proteins

The methods, compositions, kits, and cartridges of the present disclosure include a single-stranded binding protein (also abbreviated SSB or SSBP). A single-stranded binding protein may bind to a polynucleotide template strand preventing or inhibiting self-hybridization of the strand, which can prevent or inhibit formation of a secondary structure. SSBs are proteins that can bind to a single polynucleotide strand of polynucleotide. Some SSBs are sequence specific and other SSBs are not sequence specific. SSBs may have a variety of tertiary structures. In some embodiments, the SSB includes an oligonucleotide/oligosaccharide-binging fold (OB-fold), K homology domain, RNA recognition motif, whirly domain, or any combination thereof.

Any suitable SSB may be used in the methods, compositions, kits, and cartridges of the present disclosure. A suitable SSB may be an SSB is stable (e.g., thermally stable) and functional (e.g., able to bind to a single-stranded polynucleotide strand) during the one or more method steps in which it is used. An SSB may be an SSB that is found in nature or a synthetically evolved SSB. Examples of naturally occurring SSBs include those found in prokaryotes, eukaryotes, and some viruses. An SSB may be derived from a naturally occurring SSB. For example, the amino acid sequence of a naturally occurring SSB can include one or more mutations. The one or more mutations may increase the stability of the SSB, decrease the stability of the SSB, increase the binding affinity of the SSB to a single-stranded polynucleotide, decrease the binding affinity of the SSB to a single-stranded polynucleotide, increase the sequence selectivity of the SSB, decrease the sequence selectivity of the SSB, increase the expression yield of the SSB, or any combination thereof. Decreasing the affinity of an SSB to a single-stranded polynucleotide may be beneficial. For example, decreasing the affinity of an SSB to a single-stranded polynucleotide may allow for more facile removal of the SSB from a template polynucleotide strand.

Naturally occurring SSBs, and SSBs derived therefrom, as well as synthetically evolved SSBs can be produced using bacterial or eukaryotic expression systems and methods known in the art. In some embodiments, naturally occurring SSBs or an SSB derived therefrom may include a C-terminus or N-terminus purification tag. The purification tag may facilitate purification of the SSB from the expression system. Purification tags are known in the art and include, for example, a polyhistidine tag (e.g., a hexahistidine tag). The purification tag may or may not be cleaved from the SSB prior to use in the methods, compositions, kits, and cartridges of the present disclosure. For example, the purification tag may be enzymatically cleaved from the SSB.

In some embodiments, the methods, compositions, kits, and cartridges of the present disclosure include a naturally occurring prokaryotic (bacterium or archaean) SSB or an SSB derived therefrom. In some such embodiments, the SSB is an E. coli SSB, Oxytricha nova SSB, Saccharomyces cerevisiae SSB, Sulfolobus solfataricus SSB, Bacillus subtilis SSB, Caenorhabditis elegans SSB, or an SSB derived therefrom. In some embodiments, the SSB is a bacteriophage SSB or an SSB derived therefrom. In some embodiments, the SSB is a bacteriophage T4 SSB or an SSB derived therefrom.

In some embodiments, the methods, compositions, kits, and cartridges of the present disclosure include an SSB that is tolerant of and can function under extreme conditions. In some embodiments, the SSB is from a prokaryote of the Deinococcus-Thermus phylum SSB or an SSB derived therefrom. Deinococcus-Thermus prokaryotes are able to survive in harsh conditions. For example, Deinococcus-Thermus prokaryotes may be resistant to extreme heat or cold, radiation, and nuclear waste. In some embodiments, the SSB is from a prokaryote of the order Thermales or an SSB derived therefrom. In some such embodiments, the SSB is from a prokaryote of the genera Marinithermus, Meiothermus, Oceanithermus, Thermus, Vulcanithermus, Rhabdothermus, or an SSB derived therefrom. In some embodiments, the SSB is a Thermus thermophilus SSB, Thermus aquaticus SSB, or an SSB derived therefrom. In some embodiments, the SSB is from a prokaryote of the order Deinococcales or an SSB derived therefrom. In some such embodiments, the SSB is from a prokaryote of the family Deinococcaceae or an SSB derived therefrom. In some such embodiments, the SSB is from a prokaryote of the genus Deinococcus or an SSB derived therefrom. In some embodiments, the SSB is a Deinococcus radiopugnans SSB, a Deinococcus grandis SSB, a Deinococcus proteolyticus SSB, a Deinococcus murrayi SSB, or an SSB derived therefrom.

In some embodiments, the SSB is a Thermus Thermophilus (Tth) HB8 SSB or derived therefrom. In some such embodiments, the SSB includes one of the following amino acid sequences: MARGLNRVFLIGALATRPDMRYTPAGLAILDLTLAGQDLLLSDNGGEREVSWYH RVRLLGRQAEMWGDLLDQGQLVFVEGRLEYRQWEREGERRSELQIRADFLDPL DDRGKERAEDSRGQPRLRAALNQVFLMGNLTRDPELRYTPQGTAVARLGLAVN ERRQGAEERTHFVEVQAWRDLAEWAAELRKGDGLFVIGRLVNDSWTSSSGERR FQTRVEALRLERPTRGPAQAGGSRSREVQTGGVDIDEGLEDFPPEEELPF (SEQ ID NO: 1); MGSSHHHHHHSSGLVPRGSHMARGLNRVFLIGALATRPDMRYTPAGLAILDLTL AGQDLLLSDNGGEREVSWYHRVRLLGRQAEMWGDLLDQGQLVFVEGRLEYRQ WEREGERRSELQIRADFLDPLDDRGKERAEDSRGQPRLRAALNQVFLMGNLTRD PELRYTPQGTAVARLGLAVNERRQGAEERTHFVEVQAWRDLAEWAAELRKGD GLFVIGRLVNDSWTSSSGERRFQTRVEALRLERPTRGPAQAGGSRSREVQTGGV DIDEGLEDFPPEEELPF (SEQ ID NO: 2) (also termed Rev 101 and Tth SSB); GSHMARGLNRVFLIGALATRPDMRYTPAGLAILDLTLAGQDLLLSDNGGEREVS WYHRVRLLGRQAEMWGDLLDQGQLVFVEGRLEYRQWEREGERRSELQIRADF LDPLDDRGKERAEDSRGQPRLRAALNQVFLMGNLTRDPELRYTPQGTAVARLG LAVNERRQGAEERTHFVEVQAWRDLAEWAAELRKGDGLFVIGRLVNDSWTSSS GERRFQTRVEALRLERPTRGPAQAGGSRSREVQTGGVDIDEGLEDFPPEEELPF (SEQ ID NO: 3) or an amino acid sequence having 85% or greater, 90% or greater, or 95% or greater sequence identity thereto. SEQ ID NO: 2 and 3 include the amino acid sequence of SEQ ID NO: 1. SEQ ID NO: 2 includes the amino acid sequence of SEQ ID NO: 1 with an added N-terminal purification tag. SEQ ID NO: 3 is the amino acid sequence after cleavage of the N-terminal purification tag of SEQ ID NO: 2.

In some embodiments, the SSB is a mutated from of SEQ ID NO: 1. In some such embodiments has the SBS one of the following sequences: MARGLNRVFLIGALATRPDMRYTPAGLAILDLTLAGQDLLLSDNGGEREVSWYH RVRLLGRQAEMWGDLLDQGQLVFVEGRLEYRQWEREGERRSELQIRADFLDPL DDRGKERAEDSRGQPRLRAALNQVFLMGNLTRDPELRYTPQGTAVARLGLAVN ERRQGAEERTHFVEVQAWRDLAEWAAELRKGDGLFVIGRLVNDSWTSSSGERR FQTRVEALRLERPTRGPAQAGGSRSREVQTGGVDIDEGLEDPPPEEELPF (SEQ ID NO: 4); MGSSHHHHHHSSGLVPRGSHMARGLNRVFLIGALATRPDMRYTPAGLAILDLTL AGQDLLLSDNGGEREVSWYHRVRLLGRQAEMWGDLLDQGQLVFVEGRLEYRQ WEREGERRSELQIRADFLDPLDDRGKERAEDSRGQPRLRAALNQVFLMGNLTRD PELRYTPQGTAVARLGLAVNERRQGAEERTHFVEVQAWRDLAEWAAELRKGD GLFVIGRLVNDSWTSSSGERRFQTRVEALRLERPTRGPAQAGGSRSREVQTGGV DIDEGLEDPPPEEELPF (SEQ ID NO: 5) (also termed Rev 101 F225P and Tth SSB F255P); GSHMARGLNRVFLIGALATRPDMRYTPAGLAILDLTLAGQDLLLSDNGGEREVS WYHRVRLLGRQAEMWGDLLDQGQLVFVEGRLEYRQWEREGERRSELQIRADF LDPLDDRGKERAEDSRGQPRLRAALNQVFLMGNLTRDPELRYTPQGTAVARLG LAVNERRQGAEERTHFVEVQAWRDLAEWAAELRKGDGLFVIGRLVNDSWTSSS GERRFQTRVEALRLERPTRGPAQAGGSRSREVQTGGVDIDEGLEDPPPEEELPF (SEQ ID NO:6) or a sequence having 85% or greater, 90% or greater, or 95% or greater sequence identity thereto. SEQ ID NO: 5 and 6 include the amino acid sequence of SEQ ID NO: 4. SEQ ID NO: 5 includes the amino acid sequence of SEQ ID NO: 4 with an added N-terminal purification tag. SEQ ID NO: 6 is the amino acid sequence after cleavage of the N-terminal purification tag of SEQ ID NO: 5.

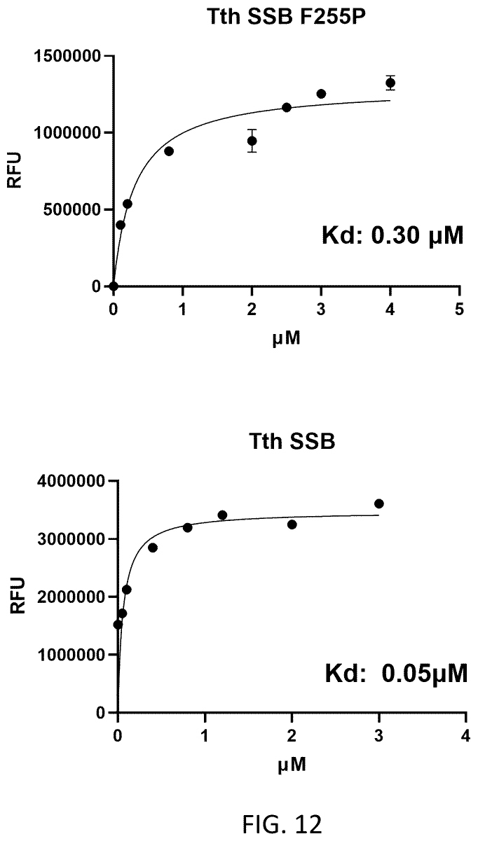

SEQ ID NO: 4 is SEQ ID NO: 1 where the phenylalanine at position 255 is mutated to proline (F255P). SEQ ID NO: 5 is SEQ ID NO: 2 where the phenylalanine at position 255 is mutated to proline (F255P). SEQ ID NO: 6 is SEQ ID NO: 3 where the phenylalanine at position 255 is mutated to proline (F255P). The F225P mutation decreases the single-stranded polynucleotide affinity sixfold. For example, the binding dissociation constant (K_{d}) of SEQ ID NO: 2 was measured to be 0.3 micromolar (μM) and the Kd of SEQ ID NO: 6 was measured to be 0.05 μM (see FIG. 12).

In some embodiments, the SSB is from a prokaryote of the Thermotogota phylum or an SSB derived therefrom. In some embodiments, the SSB is from a prokaryote of the order Thermotogales or an SSB derived therefrom. In some embodiments, the SSB is a Thermotoga martimia (Tma) SSB or an SSB derived therefrom. In some embodiments, the SSB is the Thermotoga maritimia SSB of one of the following sequences: MSFFNKIILIGRLVRDPEERYTLSGTPVTTFTIAVDRVPRKNAPDDAQTTDFFRIVT FGRLAEFARTYLTKGRLVLVEGEMRMRRWETPTGEKRVSPEVVANVVRFMDRK PAETVSETEEELEIPEEDFSSDTFSEDEPPF (SEQ ID NO:7); MGSSHHHHHHSSGLVPRGSHMSFFNKIILIGRLVRDPEERYTLSGTPVTTFTIAVD RVPRKNAPDDAQTTDFFRIVTFGRLAEFARTYLTKGRLVLVEGEMRMRRWETPT GEKRVSPEVVANVVRFMDRKPAETVSETEEELEIPEEDFSSDTFSEDEPPF (SEQ ID NO: 8) (also termed Rev 94 or Tma SSB); GSHMSFFNKIILIGRLVRDPEERYTLSGTPVTTFTIAVDRVPRKNAPDDAQTTDFF RIVTFGRLAEFARTYLTKGRLVLVEGEMRMRRWETPTGEKRVSPEVVANVVRF MDRKP (SEQ ID NO: 9) or an amino acid sequence having 85% or greater, 90% or greater, or 95% or greater sequence identity thereto. SEQ ID NO: 8 and 9 include the amino acid sequence of SEQ ID NO: 7. SEQ ID NO: 8 includes the amino acid sequence of SEQ ID NO: 7 with an added N-terminal purification tag. SEQ ID NO: 9 is the amino acid sequence after cleavage of the N-terminal purification tag of SEQ ID NO: 8.

In some embodiments, the SSB is form an archaea of the order Thermoproteales or an SSB derived therefrom. Archaea of the order Thermoproteales lack a SSB protein having the OB-fold. Instead archaea of the order Thermoproteales have a SSB termed a ThermoDBP. Generally, the ThermoDBPs tertiary structure is thought to include an extended cleft lined with phenylalanine residues and flanked by basic residues. Two ThermoDBPs homodimerize via a coiled-coil leucine zipper. In some embodiments, the SSB is a ThermoDBP SSB.

In some embodiments, the SSB is an SSB of the genus Thermoproteus, Pyrobaculum, Vulcanisaeta, Caldivirga, or an SSB derived therefrom. In some embodiments, the SSB is an SSB of the family Thermoproteus tenax, Thermoproteus thermophilus, Thermoproteus uzoniensis, or an SSB derived therefrom.

In some embodiments, the SSB is a Thermoproteus tenax SSB or derived therefrom. In some such embodiments, the SSB includes one of the following the amino acid sequences: MGEELREEERGEVRSELITKGEKKLVLIRWNTGKTSAGRLFGRYGPGGRPEFFKL LFGAVAGSLREQFGPDGENIFNRIRDSEKFRETSRELFDGLKKWFFEEAVPRYNL ERGDIFMISTELVLDPDTGELLWNRDKTQLIYWIRSDR (SEQ ID NO:10); MGSSHHHHHHSSGLVPRGSHMGEELREEERGEVRSELITKGEKKLVLIRWNTGK TSAGRLFGRYGPGGRPEFFKLLFGAVAGSLREQFGPDGENIFNRIRDSEKFRETSR ELFDGLKKWFFEEAVPRYNLERGDIFMISTELVLDPDTGELLWNRDKTQLIYWIR SDR (SEQ ID NO: 11) (also termed Rev 93); GSHMGEELREEERGEVRSELITKGEKKLVLIRWNTGKTSAGRLFGRYGPGGRPEF FKLLFGAVAGSLREQFGPDGENIFNRIRDSEKFRETSRELFDGLKKWFFEEAVPRY NLERGDIFMISTELVLDPDTGELLWNRDKTQLIYWIRSDR (SEQ ID NO: 12); or an amino acid sequence having 85% or greater, 90% or greater, or 95% or greater sequence identity thereto. SEQ ID NO: 11 and 12 include the amino acid sequence of SEQ ID NO: 10. SEQ ID NO: 11 includes the amino acid sequence of SEQ ID NO: 10 with an added N-terminal purification tag. SEQ ID NO: 12 is the amino acid sequence after cleavage of the N-terminal purification tag of SEQ ID NO: 11.

In some embodiments, the methods, compositions, kits, and cartridges of the present disclosure include a naturally occurring eukaryotic SSB or an SSB derived therefrom. In some embodiments, the SSB is a human SSB or an SSB derived therefrom.

Table 1 shows examples of naturally occurring SSBs. The methods, compositions, kits, and cartridges of the present disclosure may include any of the SSBs in Table 1 or an SSB derived therefrom. Additionally, the methods, compositions, kits, and cartridges of the present disclosure may include any of the SSBs disclosed in Chédin F, Seitz E M, Kowalczykowski S C. Novel homologs of replication protein A in archaea: implications for the evolution of ssDNA-binding proteins. Trends Biochem Sci. 1998 August; 23 (8): 273-7. doi: 10.1016/s0968-0004(98)01243-2. PMID: 9757822; Taib N, Gribaldo S, MacNeill S A. Single-Stranded DNA-Binding Proteins in the Archaea. Methods Mol Biol. 2021; 2281:23-47. doi: 10.1007/978-1-0716-1290-3_2. PMID: 33847950; or Wold MS. Replication protein A: a heterotrimeric, single-stranded DNA-binding protein required for eukaryotic DNA metabolism. Annu Rev Biochem. 1997; 66:61-92. doi: 10.1146/annurev.biochem.66.1.61. PMID: 9242902.

TABLE 1
Example single-stranded binding proteins

| Protein | Archaea/Bacteria/ | |
|---|---|---|
| Name | Eukaryote | Species |
| EcoSSB | prokaryote | E. coli |
| TEBP | eukaryote | Oxytricha nova |
| Cdc13 | eukaryote | Saccharomyces cerevisiae |
| Pot1 | eukaryote | Homo sapiens |
| ssoSSB | Archaea | Sulfolobus solfataricus |
| SsbA | Bacteria | Bacillus subtilis |
| hnRNP k | eukaryote | Homo sapiens |
| FBP1 | eukaryote | Homo sapiens |
| FBP2 | eukaryote | Homo sapiens |
| FBP3 | eukaryote | Homo sapiens |
| FBP4 | eukaryote | Homo sapiens |
| PCBP1 | eukaryote | Homo sapiens |
| PCBP2 | eukaryote | Homo sapiens |
| UP1 | eukaryote | Homo sapiens |
| RBM-45 | eukaryote | Homo sapiens |
| FIR | eukaryote | Homo sapiens |
| SUP-12 | Eukaryote | Caenorhabditis elegans |
| MEC-8 | Eukaryote | Caenorhabditis elegans |
| RPA | Eukaryote | multiple |
| | (Mitocrhondia) | |
| mtSSB | Bacteria | Homo sapiens |
| TmaSSB | Bacteria | Thermotoga maritima |
| (SEQ ID | | |
| NO: 3A) | | |
| TneSSB | Bacteria | Thermus thermophilus |
| TthSSB | Bacteria | Thermus thermophilus |
| (SEQ ID | | |
| NO: 1A) | | |
| TaqSSB | Bacteria | Thermus aquaticus |
| Ssb | Bacteria | Deinococcus radiodurans |
| ddrB | Bacteria | Deinococcus radiodurans |
| DrpSSB | Bacteriae | Deinococcus radiopugnans |
| DrgSSB | Bacteria | Deinococcus grandis |
| DprSSB | Bacteria | Deinococcus proteolyticus |
| DmuSSB | Bacteria | Deinococcus murrayi |

### Single-Stranded Sequencing Methods and Compositions

For simplicity, the methods of the present disclosure are described relative to sequencing a single polynucleotide template strand. It is understood that the methods may be used to sequence two or more polynucleotide template strands simultaneously. For example, sequencing methods described herein may be applied to arrays or clusters of polynucleotide template stands in order to accomplish massive parallel sequencing.

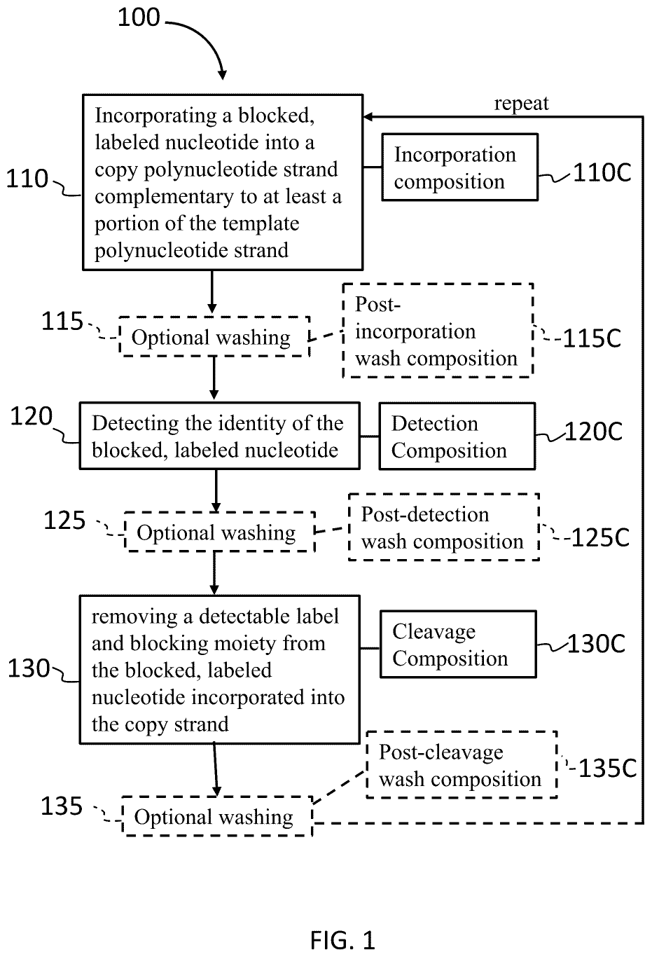

FIG. 1 is a flow chart illustrating an overview of a polynucleotide sequencing method 100 consistent with some embodiments of the present disclosure. The method 100 includes sequencing by synthesis.

Briefly, sequencing by synthesis employs a number of sequencing by synthesis reactions to elucidate the identity of a plurality of bases at target positions within a target sequence. All these reactions rely on the use of a target nucleic acid sequence (polynucleotide template) having at least two domains; a first domain to which a sequencing primer will hybridize, and an adjacent second domain, for which sequence information is desired. Upon formation of an assay complex, extension enzymes are used to add deoxynucleotide triphosphates (dNTPs) to the sequencing primer that is hybridized to the first domain, and each addition of dNTPs is read to determine the identity of the added dNTP. This may proceed for many cycles. Sequencing by synthesis techniques such as, the Genome Analyzer systems (Illumina Inc., San Diego, CA) and the True Single Molecule Sequencing (tSMS) systems (Helicos BioSciences Corporation, Cambridge, MA), utilize labeled nucleotides to determine the sequence of a target nucleic acid molecule (polynucleotide template). A target nucleic acid molecule (polynucleotide template) can be hybridized with a primer and incubated in the presence of a polymerase and a labeled nucleotide containing a blocking group. The primer is extended such that the nucleotide is incorporated. The presence of the blocking group permits only one round of incorporation, that is, the incorporation of a single nucleotide. The presence of the label permits identification of the incorporated nucleotide. A plurality of homogenous single nucleotide bases can be added during each cycle, such as used in the True Single Molecule Sequencing (tSMS) systems (Helicos BioSciences Corporation, Cambridge, MA). Alternatively, all four nucleotide bases can be added during each cycle simultaneously, such as used in the Genome Analyzer systems (Illumina Inc., San Diego, CA), particularly when each base is associated with a distinguishable label. After identifying the incorporated nucleotide by its corresponding label, both the label and the blocking group can be removed, thereby allowing a subsequent round of incorporation and identification. Determining the identity of the added nucleotide base includes, in some embodiments, repeated exposure of the newly added labeled bases to a light source that can induce a detectable emission due to the addition of a specific nucleotide base, i.e. dATP, dCTP, dGTP or dTTP. The methods and compositions disclosed herein are particularly useful for such SBS techniques. In addition, the methods and compositions described herein may be particularly useful for sequencing from an array of nucleic acids, where multiple sequences can be read simultaneously from multiple positions on the array since each nucleotide at each position can be identified based on its identifiable label. Exemplary methods are described in US 2009/0088327; US 2010/0028885; and US 2009/0325172, each of which is incorporated herein by reference.

Referring back to FIG. 1, overviews of some steps in the SBS process of the methods of the present disclosure are shown. The compositions employed at different method steps of the SBS process are also shown. The method 100 includes incorporating a blocked, labeled nucleotide into a copy nucleotide strand, the copy nucleotide strand complementary to and hybridized with at least a portion of a template polynucleotide strand (step 110; also referred to as an incorporation step). The method 100 further includes detecting the identity of the blocked, labeled nucleotide (step 120; also referred to as a detection step). The method 100 further includes removing a detectable label and blocking group from the blocked, labeled, nucleotide that was previously incorporated into the copy polynucleotide strand (step 130; also referred to as a cleavage step). The incorporation, detection, and cleavage steps may be repeated any number of times to sequence at least a portion of the template polynucleotide strand. In some embodiments, a washing step may be employed after one or more steps. For example, the product of the previous step in the method may be exposed to a wash composition (e.g., step 115, 125, and 135).

The method steps may be repeated any number of times. For example, the method steps may be repeated to allow for 2 to 500, 2 to 400, 2 to 300, 2 to 200, 2 to 100, 2 to 50, 25 to 300, 25 to 100, or 25 to 50 incorporation and detection cycles.

Throughout the method, the template polynucleotide strand and the copy nucleotide strand form a template polynucleotide strand-copy nucleotide strand complex, also termed a sequencing complex. At the locations in which the template polynucleotide strand is complementary to and hybridized with the copy polynucleotide strand, the sequence complex is double stranded. As the method steps are repeated, the length of the double stranded region of the sequencing complex grows as nucleotides are added to the copy polynucleotide strand.

At least one of the incorporation step, detection step, and cleavage step is performed in the presence of a single-stranded binding protein. The single-stranded binding protein may be any suitable single-stranded binding protein such as those described herein. In some embodiments, the incorporation step (step 110) is performed in the presence of a single-stranded binding protein. In some embodiments, the optional washing step following the incorporation step (step 115) is performed in the presence of a single-stranded binding protein. In some embodiments, the detection step (step 120) is performed in the presence of a single-stranded binding protein. In some embodiments, the optional washing step following the detection step (step 125) is performed in the presence of a single-stranded binding protein. In some embodiments, the cleavage step (step 130) is performed in the presence of a single-stranded binding protein. In some embodiments, the optional washing step following the cleavage step (step 135) is performed in the presence of a single-stranded binding protein.

In some embodiments, the incorporation step (step 110) and the detection step (step 120) are performed in the presence of a single-stranded binding protein. In some embodiments, the incorporation step (step 110) and the cleavage step (step 130) are performed in the presence of a single-stranded binding protein. In some embodiments, the detection step (step 120) and the cleavage step (step 130) are performed in the presence of a single-stranded binding protein. In some embodiments, the incorporation step (step 110), the detection step (step 120), and the cleavage step (step 130) are performed in the presence of a single-stranded binding protein.

The steps of the method 100 may be performed in the presence of a method composition. A method composition may include the reagents used to accomplish a method step. The term “method composition” includes any composition used in a sequencing method, including, for example, an incorporation composition (110C), detection composition (120C), cleavage composition (130C), and a wash composition (post-incorporation wash composition (115C), post-detection wash composition (125C), post-cleavage wash composition (135C). For example, the incorporation step (step 110) may be accomplished in the presence of an incorporation composition (110C). The detection step (step 120) may be accomplished in the presence of a detection composition (120C). The deblocking step (step 130) may be accomplished in the presence of a cleavage composition (130C). In embodiments, where a washing step is employed before or after one or more of the steps, a wash composition is used.

In embodiments where the step is performed in the presence of a single-stranded binding protein, the method composition used to accomplish the step includes the single-stranded binding protein. For example, in embodiments were the incorporation step (step 110) is performed in the presence of a single-stranded binding protein, the incorporation composition (110C) includes the single-stranded binding protein. In embodiments were the optional post-incorporation washing step (step 115) is performed in the presence of a single-stranded binding protein, the post-incorporation composition (115C) includes the single-stranded binding protein. In embodiments were the detection step (step 120) is performed in the presence of a single-stranded binding protein, the detection composition (120C) includes the single-stranded binding protein. In embodiments were the optional post-detection washing step (step 125) is performed in the presence of a single-stranded binding protein, the post-detection wash composition (125C) includes the single-stranded binding protein. In embodiments were the cleavage step (step 130) is performed in the presence of a single-stranded binding protein, the cleavage composition (130C) includes the single-stranded binding protein. In embodiments were the optional post-cleavage step (step 135) is performed in the presence of a single-stranded binding protein, the post-cleavage composition (135C) includes the single-stranded binding protein.

It will be understood that any method composition may be incubated with a sequencing complex for a period of time rather than continuously flowing the method composition passed the sequencing complex. Of course, any method composition may be continuously flowed passed the sequencing complex.

The amount of single-stranded binding protein in a method composition may vary. In some embodiments a method composition includes 0.01 milligrams per milliliter (mg/ml) or greater, 0.05 mg/ml or greater, 0.1 mg/ml or greater, 0.2 mg/ml or greater, 0.3 mg/ml or greater, 0.5 mg/ml or greater, 0.6 mg/ml or greater, 0.7 mg/ml or greater, 0.8 mg/ml or greater, 0.9 mg/ml or greater, 1 mg/ml or greater, or 1.5 mg/ml or greater single-stranded binding protein. In some embodiments, a method composition includes 2 mg/ml or less, 1.5 mg/ml or less, 1 mg/ml or less, 0.9 mg/ml or less, 0.8 mg/ml or less, 0.7 mg/ml or less, 0.6 mg·ml or less, 0.5 mg/ml or less, 0.4 mg/ml or less, 0.3 mg/ml or less, 0.2 mg/ml or less, 0.1 mg/ml or less, or 0.05 mg/ml or less single-stranded binding protein. In some embodiments, method composition includes 0.01 mg/ml to 1 mg/ml, 0.1 mg/ml to 0.5 mg/ml or 0.1 mg/ml to 0.3 mg/ml single-stranded binding protein.

In some embodiments, the incorporation step (step 110) is performed in the presence of a single-stranded binding protein. In some such embodiments, the incorporation composition (110C) includes 0.01 mg/ml to 1 mg/ml, 0.1 mg/ml to 0.5 mg/ml, or 0.1 mg/ml to 0.3 mg/ml single-stranded binding protein.

In some embodiments, the optional post-incorporation wash step (step 115) is performed in the presence of a single-stranded binding protein. In some such embodiments, the post-incorporation wash composition (115C) includes 0.01 mg/ml to 1 mg/ml, 0.1 mg/ml to 0.5 mg/ml, or 0.1 mg/ml to 0.3 mg/ml single-stranded binding protein.

In some embodiments, the detecting step (step 120) is performed in the presence of a single-stranded binding protein. In some such embodiments, the detection composition (120C) includes 0.01 mg/ml to 1 mg/ml, 0.1 mg/ml to 0.5 mg/ml, or 0.1 mg/ml to 0.3 mg/ml single-stranded binding protein.

In some embodiments, the optional post-detection wash step (step 125) is performed in the presence of a single-stranded binding protein. In some such embodiments, the post-detection wash composition (125C) includes 0.01 mg/ml to 1 mg/ml, 0.1 mg/ml to 0.5 mg/ml, or 0.1 mg/ml to 0.3 mg/ml single-stranded binding protein.

In some embodiments, the cleavage step (step 130) is performed in the presence of a single-stranded binding protein. In some such embodiments, the cleavage composition (130C) includes 0.01 mg/ml to 1 mg/ml, 0.1 mg/ml to 0.5 mg/ml, or 0.1 mg/ml to 0.3 mg/ml single-stranded binding protein.

In some embodiments, the optional post-cleavage wash step (step 135) is performed in the presence of a single-stranded binding protein. In some such embodiments, the post-cleavage wash composition (125C) includes 0.01 mg/ml to 1 mg/ml, 0.1 mg/ml to 0.5 mg/ml, or 0.1 mg/ml to 0.3 mg/ml single-stranded binding protein.

The components of a method composition are dependent at least in part on the method step in which they are employed. Any method composition may include a salt, a buffer, a detergent, a chelating agent, an antioxidant, scavenger, a single-stranded binding protein or any combination thereof.

In some embodiments, a method composition includes a salt. Examples of salts include sodium chloride, potassium chloride, and lithium chloride. The salt may be present in a method composition at any suitable concentration. For example, the salt may be present at a concentration from 10 millimolar (mM) to 250 mM, such as from 25 mM to 100 mM, 30 mM to 70 mM, or 50 mM.

In some embodiments, one or more of the sequencing method steps are performed in the absence of potassium ions. That is, one or more of the method compositions are free of potassium salts that can dissociate to form potassium ions. Potassium ions are able to stabilize secondary structures such as g-quadruplexes. As such, it may be advantageous to perform one or more of the sequencing method steps in the absence of potassium ions. In some such embodiments, a method composition may not include a salt, or may include a non-potassium containing salt such as lithium chloride.

In some embodiments, a method composition includes both a non-potassium containing salt and a single-stranded binding protein. In other embodiments, a method composition includes a non-potassium containing salt but does not include a single-stranded binding protein.

In some embodiments, the incorporation step (step 110) is performed in the absence of potassium ions. In some such embodiments, the incorporation composition (110C) does not include a salt or includes a non-potassium containing salt.

In some embodiments, the optional post-incorporation wash step (step 115) is performed in the absence of potassium ions. In some such embodiments, the post-incorporation wash composition (115C) does not include a salt or includes a non-potassium containing salt.

In some embodiments, the detection step (step 120) is performed in the absence of potassium ions. In some such embodiments, the detection composition (120C) does not include a salt or includes a non-potassium containing salt.

In some embodiments, the optional post-detection wash step (step 125) is performed in the absence of potassium ions. In some such embodiments, the post-detection wash composition (125C) does not include a salt or includes a non-potassium containing salt.

In some embodiments, the cleavage step (step 130) is performed in the absence of potassium ions. In some such embodiments, the cleavage composition (130C) does not include a salt or includes a non-potassium containing salt.

In some embodiments, the optional post-cleavage wash step (step 135) is performed in the absence of potassium ions. In some such embodiments, the post-cleavage wash composition (135C) does not include a salt or includes a non-potassium containing salt.

In some embodiments, a method composition includes a buffer. For example, one or more of an incorporation composition (110C), a post-incorporation wash composition (115C), a detection composition (120C), a post-detection wash composition (125C), a cleavage composition (130), and a post-cleavage wash composition (135C), may each independently include a buffer. An example of a buffer includes a tris (hydroxymethyl) aminomethane (Tris) buffer. The amount of the buffer in a method composition may vary. For example, the buffer may be present at a concentration of 5 mM to 2 molar (M) such as 10 mm to 1.5 M, or 50 mM to 1 M. In some preferred embodiments, a method composition includes a Tris buffer at a concentration of 75 mM to 250 mM, such from 100 mM to 200 mM, or 150 mM.

In some embodiments, a method composition includes a detergent. For example, one or more of an incorporation composition (110C), a post-incorporation wash composition (115C), a detection composition (120C), a post-detection wash composition (125C), a cleavage composition (130), and a post-cleavage wash composition (135C), may each independently include a detergent. Any suitable detergent may be included in a method composition. For example, a method composition may include an anionic, cationic, zwitterionic or nonionic detergent. In some preferred embodiments, a method composition includes a nonionic detergent. An example of a suitable nonionic detergent is Tween 20 (available from ThermoFischer Scientific). The detergent may be present in a method composition at any suitable concentration. For example, the detergent may be present in a method composition from 0.01% by weight to 0.5% by weight, such as 0.02% by weight to 0.1% by weight, or 0.03% by weight to 0.07% by weight. In some preferred embodiments, method composition comprises Tween 20 at a concentration of 0.03% by weight to 0.07% by weight, or 0.5% by weight.

In some embodiments, a method composition includes a chelating agent. For example, one or more of an incorporation composition (110C), a post-incorporation wash composition (115C), a detection composition (120C), a post-detection wash composition (125C), a cleavage composition (130), and a post-cleavage wash composition (135C), may each independently include a chelating agent. Any suitable chelating agent may be included in a method composition. For example, a method composition may include dihydroxyethylglycine (HEG) or ethylenediaminetetraacetic acid (EDTA). The chelating agent may be present in any suitable concentration. For example, the chelating agent may be present a method composition at a concentration from 0.1 mM to 50 mM, such as 0.5 mM to 20 mM. In some preferred embodiments, a method composition includes HEG at a concentration from 5 mM to 15 mM, such as 10 mM.

In some embodiments, a method composition includes an antioxidant. An antioxidant may be included to prevent photo-induced damage. For example, one or more of an incorporation composition (110C), a post-incorporation wash composition (115C), a detection composition (120C), a post-detection wash composition (125C), a cleavage composition (130), and a post-cleavage wash composition (135C), may each independently include an antioxidant. The composition may comprise any suitable amount of an antioxidant. For example, the composition may comprise one or more antioxidant in a combined total antioxidant concentration from about 2 mM to about 50 mM, such as from about 5 mM to about 40 mM, or from about 15 mM to about 25 mM, or about 20 mM. Suitable antioxidants include ascorbate, acetovanillone, and 6-hydroxy-2,5,7,8-tetramethylchroman-2-carboxylic acid (Trolox). In some preferred embodiments, the wash composition comprises sodium ascorbate.

In some embodiments, a method composition includes a scavenger. For example, one or more of an incorporation composition (110C), a post-incorporation wash composition (115C), a detection composition (120C), a post-detection wash composition (125C), a cleavage composition (130), and a post-cleavage wash composition (135C), may each independently include a scavenger. As used herein, a “scavenger” is a compound that inhibits interaction of reactive compounds used in or resulting from the cleavage step (step 130) with polynucleotides used in or generated from the sequencing process, enzymes used in the sequencing process, or other reagents or compounds used in the sequencing process. In some embodiments, a scavenger is a compound that oxidizes a cleavage agent that is a reducing agent under conditions of the sequencing procedure. Any suitable scavenger may be used. Examples of scavengers include cystine, lipoic acid, 3,3′-dithiodipropionic acid (DPPA), and a pegylated azide OH—(CH₂CH₂O)ₙCH₂CH₂—N₃. The scavenger may be present in a method composition at any suitable concentration. For example, the scavenger may be present in a method composition at a concentration from 0.1 mM to 50 mM, such as from 0.5 mM to 20 mM, or from 1 mM to 10 mM.

In some embodiments, one or more of the method compositions may be the same. For example, in some embodiments the post-cleavage wash composition (135C) and the detection composition (120C) may be the same. When the same composition is used for multiple sequencing steps, the number of compositions used in the sequencing process may be reduced, which may provide one or more advantages. For example, reducing the number of reagents or method compositions may reduce the burden of ensuring good manufacturing practices and method composition or reagent stability. The cartridge size and complexity of a sequencing instrument used to carry out the sequencing method may be reduced. There may be fewer failure modes, and there may be savings in the cost of goods. These and other advantages will be evident to those of skill in the polynucleotide sequencing arts.

Referring back to FIG. 1, the incorporation step (110) may include the use of an incorporation composition (110C). For example, step 110 may include exposing a sequencing complex (i.e., template polynucleotide strand-copy nucleotide strand complex) to an incorporation composition 110C. The incorporation composition includes at least some of the components for the addition of the block, labeled nucleotide (e.g., a blocked and labeled, dATP, dCTP, dGTP, or dTTP) to the copy polynucleotide strand. For example, the incorporation composition includes the blocked, labeled nucleotide or a plurality of blocked, labeled nucleotides. In some embodiments, the incorporation composition includes a polymerase. The polymerase catalyzes the incorporation of the blocked, labeled nucleotide into the copy polynucleotide strand. Any suitable polymerase may be used including those described in U.S. Provisional Patent Application No. 63/412,241; US Patent Application Number U.S. Ser. No. 16/703,569 (U.S. Pat. No. 11,001,816B2), and PCT Application Number PCT/US2013/03169 (WO2014142921A1). The incorporation composition may also include a salt, a buffer, a chelating agent, an antioxidant, a scavenger, a single-stranded binding protein, or any combination thereof such as described herein.

The copy polynucleotide strand is synthesized through a sequencing by synthesis process. For example, a sequencing primer hybridizes to a portion of the template polynucleotide strand and the is extended through the incorporation of blocked, labeled nucleotides. In some embodiments, SBS involves several rounds of incorporation of nucleotides for which the identity of the incorporated nucleotides are not determined. Such rounds of incorporation may be referred to as “dark cycles.” Dark cycling involves the sequential incorporation of nucleotides containing a 3′ blocking group and subsequent blocking group removal. Dark cycles may be used to skip the reading of index sequences, universal sequences, and/or any other sequence where the identity is not desired to be determined.

The blocked, labeled nucleotide includes a 3′ blocking group and a detectable label. The blocking group inhibits 5′ to 3′ chain extension allowing the incorporation of a single nucleotide. A blocking group is removable to expose 3′ end of the polynucleotide to allow for future nucleotide incorporation. The blocked, labeled nucleotide may include any suitable blocking group.

The skilled person will appreciate how to attach a suitable blocking group to a ribose ring of a nucleotide to block interactions with 3′-OH. The blocking group may be attached directly at 3′ position or may be attached at the 2′ position (the blocking group being of sufficient size or charge to block interactions at 3′ position). Alternatively, the blocking group may be attached at both 3′ and 2′ positions and may be cleaved to expose 3′ OH group.

Suitable blocking groups will be apparent to the skilled person and may be formed from any suitable protecting group disclosed, for example, in “Protective Groups in Organic Synthesis”, T. W. Greene and P. G. M. Wuts, 3rd Ed., Wiley Interscience, New York, which is hereby incorporated herein by reference in its entirety to the extent that it does not conflict with the present disclosure. The blocking group is preferably removable (or modifiable) to produce a 3′ OH group. The process used to obtain 3′ OH group may be any suitable chemical or enzymic reaction.

Blocking moieties may be as described in U.S. Pat. No. 7,414,116, which is hereby incorporated herein by reference in its entirety to the extent that it does not conflict with the present disclosure

The blocked, labeled nucleotide includes a detectable label. Any detectable label may be used. For example, the detectable label may be a fluorophore, quantum dot, gold nanoparticle, or microbead. Detection can be carried out by any suitable method, including fluorescence spectroscopy or by other optical means. In some embodiments, the detectable label includes a fluorophore. Fluorophores absorb energy at an excitation wavelength and after absorption, emit radiation at an emission wavelength. Each nucleotide (i.e., A, T, G, C) may include a different fluorophore that have distinctive emission wavelengths allowing for identification of the nucleotide incorporated into the copy polynucleotide strand.

The blocking moiety, the labeled moiety, or the blocking moiety and the labeled moiety molecule may be linked to the nucleotide by any suitable linker. The linker may comprise one or more cleavable groups including, but not limited to, disulfide, diol, diazo, ester, sulfone azide, alyl and silyl ether, azide and alkoxy groups. In preferred embodiments, the linker comprises one or more of an azide, an alkoxy, and a disulfide group as a linker. Incorporation of a disulfide bond into a linker may be accomplished in a number of ways, for example as described in U.S. Pat. No. 7,771,973 or as described in Hermanson, Bioconjugate Techniques, Second Edition, Academic Press (incorporated herein by reference in their entireties).

More generally, suitable linkers include, but are not limited to, disulfide linkers, acid labile linkers (including dialkoxybenzyl linkers, Sieber linkers, indole linkers, and t-butyl Sieber linkers), electrophilically cleavable linkers, nucleophilically cleavable linkers, photocleavable linkers, cleavage under reductive conditions, oxidative conditions, cleavage via use of safety-catch linkers, and cleavage by elimination mechanisms.

Any suitable electrophilically cleavable linkers may be employed. Electrophilically cleavable linkers are typically cleaved by protons and include cleavages sensitive to acids. Suitable electrophilically cleavable linkers include the modified benzylic systems such as trityl, p-alkoxybenzyl esters and p-alkoxybenzyl amides. Other suitable electrophilically cleavable linkers include tert-butyloxycarbonyl (Boc) groups and the acetal system.

The use of thiophilic metals, such as nickel, silver or mercury, in the cleavage of thioacetal or other sulfur-containing protecting groups can also be considered for the preparation of suitable electrophilically cleavable linkers molecules.

Any suitable nucleophilic cleavage linker may be employed. Nucleophilic cleavage is a well-recognized method in the preparation of linker molecules. Groups such as esters that are labile in water (i.e., can be cleaved simply at basic pH) and groups that are labile to non-aqueous nucleophiles, may be used. Fluoride ions may be used to cleave silicon-oxygen bonds in groups such as triisopropyl silane (TIPS) or t-butyldimethyl silane (TBDMS).

Any suitable photocleavable linker may be used. Photocleavable linkers have been used widely in carbohydrate chemistry. It is preferable that the light required to activate cleavage does not affect the other components of the modified nucleotides. For example, if a fluorophore is used as the label, it is preferable if this absorbs light of a different wavelength to that required to cleave the linker molecule. Suitable linkers include those based on O-nitrobenzyl compounds and nitroveratryl compounds. Linkers based on benzoin chemistry may also be used (Lee et al., J. Org. Chem. 64:3454-3460, 1999).

Any suitable linker that cleaves under reductive conditions may be used. There are known many linkers that are susceptible to reductive cleavage. For example, catalytic hydrogenation using palladium-based catalysts has been used to cleave benzyl and benzyloxycarbonyl groups. By way of further example, disulfide bond reduction is also known in the art.

Any suitable linker that cleaves under oxidative conditions may be used. Oxidation-based approaches are well known in the art. These include oxidation of p-alkoxybenzyl groups and the oxidation of sulfur and selenium linkers. The use of aqueous iodine to cleave disulfides and other sulfur or selenium-based linkers is also within the scope of the invention.

Any suitable safety-catch linker may be used. Safety-catch linkers are those that cleave in two steps. In a preferred system, the first step is the generation of a reactive nucleophilic center followed by a second step involving an intra-molecular cyclization that results in cleavage. For example, levulinic ester linkages may be treated with hydrazine or photochemistry to release an active amine, which may then be cyclized to cleave an ester elsewhere in the molecule (Burgess et al., J. Org. Chem. 62:5165-5168, 1997).

Any suitable linker that may be cleaved by elimination mechanisms may be used. For example, the base-catalyzed elimination of groups such as Fmoc and cyanoethyl, and palladium-catalyzed reductive elimination of allylic systems, may be used.

The linkers may include one or more spacer in addition to the cleavage site. The spacer distances e.g., the nucleotide base from the cleavage site or label or blocking moiety. The length of the linker is generally not important provided that the nucleotide may be incorporated into the copy strand after by a chain extending enzyme after the blocking moiety is cleaved.

Examples of suitable linkers, nucleotides, blocking moieties that may be employed are described in U.S. Pat. No. 7,541,444; WO 03/048387; US 2013/0079232A1; and U.S. Pat. No. 7,414,116, each of which is hereby incorporated herein in their respective entireties to the extent that they do not conflict with the present disclosure. Particularly preferred linkers are phosphine-cleavable azide containing linkers. The labeled moiety may comprise a fluorophore.

In some embodiments, the method 100 includes washing away any unincorporated blocked, labeled, nucleotides. Unincorporated blocked, labeled nucleotides are nucleotides that were present in the incorporation composition but not covalently coupled to the copy polynucleotide strand during the incorporation step. Washing away unincorporated blocked, labeled, nucleotides may be accomplished with a post-incorporation wash composition. For example, the sequencing complex may be exposed to a post-incorporation wash composition.

The method 100 further includes detecting the identity of the blocked, labeled nucleotide (120). Determining the identity of the added nucleotide base includes, in some embodiments, exposure of the sequencing complex to a radiation source. Exposure of the sequencing complex to a radiation source that can excite the detectable label on the newly incorporated blocked, detectable label. The excited detectable label will relax emitting energy at an emission wavelength. The emission wavelength can be detected, for example, using spectroscopy such as fluorescence spectroscopy.

Detection of the blocked, labeled nucleotide (step 120) may be accomplished in the presence of a detection composition (120C), also called a scanning composition. For example, the sequencing complex may be exposed to a detection composition followed by exposure to a radiation source. The scanning composition may include an antioxidant. The antioxidant may protect the polynucleotide template strand and the copy polynucleotide strand from damage that may be induced by light during the detection step (See, e.g., U.S. Pat. Nos. 9,115,353 and 9,217,178). In some embodiments, a post-incorporation wash composition (115C) may be employed to wash away the unincorporated blocked, labeled nucleotides prior to introduction of the detection composition. In other embodiments, the introduction of the detection composition (120C) may serve to wash away the unincorporated blocked, labeled nucleotides.

In some embodiments, the detection composition includes a single-stranded binding protein. Inclusion of a single-stranded binding protein in the detection composition may protect the polynucleotide template strand and/or the copy polynucleotide strand from degradation caused by radiation exposure. As such, the use of a single-stranded binding protein in a detection composition may enable longer runs (repetition of the sequencing steps to incorporate and detect multiple nucleotides into the copy polynucleotide strand), as damage to the polynucleotides may be reduced relative to sequencing processes that do not perform a detection step in the presence of a single-stranded binding protein.

Inclusion of a single-stranded binding protein in the detection composition may allow for the use of a higher amount of radiation energy to be used during the detection step. For example, in some cases, increased radiation energy may be used to increase the signal to noise ratio when sequencing fewer polynucleotide template strands. The inclusion of a single-stranded binding protein in the detection composition may protect the polynucleotide template strand and/or the copy polynucleotide strand from degradation caused by the increased radiation exposure.

The method 100 further includes removing a detectable label and blocking moiety from the blocked, labeled nucleotide incorporated into the copy strand (step 130). In some embodiments, the detectable label and blacking moiety are chemically removed from the blocked, labeled nucleotide. The cleavage step (also called a de-blocking step) may be accomplished in the presence of a cleavage composition (130C) also called a de-blocking composition. In embodiments where the cleavage step is a chemical cleavage step, the cleavage composition includes a cleavage agent. Preferably, cleavage agent removes both the blocking group and the detectable label. For example, the labeled moiety may serve as the blocking group, the detectable label may be on the blocking group, the detectable label moiety may be attached to the nucleotide by the same linker as the blocking group, etc.

The type of cleavage agent used is dependent at least in part on the cleavage group present. For example, cleavage of disulfide bonds or other reductive cleavage groups may be accomplished by a reducing agent. Reduction of a disulfide bond results in the release of the linked molecule from the nucleotide. Reducing agents useful in practicing embodiments as described herein include, but are not limited to, phosphine compounds, water soluble phosphines, nitrogen containing phosphines and salts and derivatives thereof, dithioerythritol (DTE), dithiothreitol (DTT) (cis and trans isomers, respectively, of 2,3-dihydroxy-1,4-dithiolbutane), 2-mercaptoethanol or β-mercaptoethanol (BME), 2-mercaptoethanol or aminoethanethiol, glutathione, thioglycolate or thioglycolic acid, 2,3-dimercaptopropanol and tris (2-carboxyethyl) phosphine (TCEP), tris (hydroxymethyl) phosphine (THP) and β-[tris (hydroxymethyl) phosphine] propionic acid (THPP). In some embodiments, a reducing agent used for cleaving a disulphide bond in a linker as described herein is DTT. In some embodiments, the concentration of a reducing reagent, for example DTT, utilized for cleaving a disulfide bond is at least 1 to 1000 mM, at least 20 to 800 mM, at least 40 to 500 mM, and preferably at least 50 to 200 mM.

In some embodiments, a reducing agent used for cleaving a disulphide bond in a linker or a cleavable linker comprising an allyl or azido group is a phosphine reagent, a water-soluble phosphine reagent, a nitrogen containing phosphine reagent and salts and derivatives thereof. Exemplary phosphine reagents include, but are not limited to, tris (2-carboxyethyl) phosphine (TCEP), tris (hydroxypropyl) phosphine (THP), tris (hydroxymethyl) phosphine (TMP) and those disclosed in US patent publication 2009/0325172 (incorporated herein by reference in its entirety) such as triaryl phosphines, trialkyl phosphines, sulfonate containing and carboxylate containing phosphines and derivatized water soluble phosphines. Other phosphines that may be used as cleavage agents include those described in U.S. Pat. No. 7,414,116, which is hereby incorporated herein by reference in its entirety to the extent that it does not conflict with the present disclosure. In some embodiments, the concentration of a phosphine utilized is from 0.5 mM to 500 mM, such as from 5 mM to 50 mM, and preferably from 10 mM to 40 mM. Methods and compositions as described herein are not limited by any particular cleavage group and alternatives will be readily apparent to a skilled artisan and are considered within the scope of the present disclosure.

In some embodiments, the method 100 further includes washing away the cleaved blocking group and cleaved detectable label. Washing away the cleaved detectable label and cleaved blocking group may be accomplished with a post-cleavage wash composition. For example, the sequencing complex may be exposed to a post-cleavage wash composition. In some embodiments, the post-cleavage wash composition includes a scavenger compound.

The method 100 of the present disclosure may be used to simultaneously sequence a plurality of polynucleotide templates. The method 100 of the present disclosure may be repeated to simultaneously sequence a plurality of polynucleotide templates. The sequential completion of an incorporation step, a detection step, and a cleavage step may be referred to as a cycle. Sequencing of a polynucleotide template or a plurality of polynucleotide templates may include completing 1 or more cycles, 5 or more cycles, 10 or more cycles, 25 or more cycles, 50 or more cycles, 75 or more cycles, 100 or more cycles, 125 or more cycles, 150 or more cycles, 200 or more cycles, 250 or more cycles, or 300 or more cycles.

In some embodiments, the method 100 may be a method for improving secondary structure resolution of a polynucleotide sequencing by synthesis process. For example, in some embodiments, the method 100 may be a method for improving secondary structure resolution during sequencing by synthesis of a polynucleotide template. Specifically, the method 100 that includes performing one or more steps in the presence of a single-stranded binding protein may be a method for improving secondary structure resolution during sequencing of a polynucleotide template as compared to sequencing the same polynucleotide template using the same method but without the use of single-stranded binding protein. In some embodiments, the method (100) that includes the use of a single-stranded binding protein may be a method for improving g-quadruplex resolution during sequencing of a polynucleotide template as compared to sequencing the same polynucleotide template using method 100 but without the use of a single-stranded binding protein.

Secondary structure resolution is the ability to sequence through one or more secondary structure such as a G-quadruplex. The methods of the present disclosure that perform one or more steps in the presence of a single-stranded binding protein, which may improve secondary structure resolution compared to the same method performed without a single-stranded binding protein when both methods are used to sequence the same polynucleotide template. Secondary structure resolution can be quantified using a resolution value. Secondary structure resolution is generally determined using a plurality of polynucleotides templates of the same known sequence and known regions of secondary structure. A resolution value is the percent of nucleotides correctly identified in one or more regions of known secondary structure of a polynucleotide template normalized to 100% when a plurality of polynucleotide templates of the same, known sequence are sequenced.

In some embodiments, a method of the present disclosure, in which at least one step of the method is performed in the presence of a single-stranded binding protein, results in a resolution value that is greater than the resolution value of the same method performed in the absence of the single-stranded binding protein when both methods are used to sequence a plurality of the same polynucleotide template. In some embodiments, the resolution value of a method where at least one step is performed in the presence of a single-stranded binding protein is 1% or greater, 5% or greater, 7% or greater, 10% or greater, 15% or greater, 20% or greater, 25% or greater, 30% or greater, 35% or greater, 40% or greater, 45% or greater, or 50% or greater than the resolution value of the same method performed without the single-stranded binding protein when both methods are used to sequence a plurality of the same polynucleotide template.

In some embodiments, the method 100 may be a method for lowering the error rate of a polynucleotide sequencing by synthesis process. For example, in some embodiments, the method (100) may be a method for lowering the error rate during sequencing of polynucleotide templates. Specifically, the method 100 that includes the use of a single-stranded binding protein may be a method for lowering the error rate during sequencing of a polynucleotide template as compared to sequencing the same polynucleotide template using method 100 without a single-stranded binding protein.

Error rate is the percent of nucleotides incorrectly identified when sequencing a plurality of polynucleotide templates of the same sequence of a known sequence. For example, an error rate of 5% indicates that 5% of nucleotides are identified incorrectly. The error rate may vary depending on the number of sequencing cycles. For example, the error rate may be 3% at cycle 10 and 5% at cycle 50. Generally, the error rate increases as the number of cycles increase.

The method 100 of the present disclosure that performs one or more steps in the presence of a single-stranded binding protein may have a lower error rate compared to the same method performed without a single-stranded binding protein when both methods are used to sequence a plurality of the same polynucleotide template. The method 100 of the present disclosure that performs one or more steps in the presence of a single-stranded binding protein may have a lower error rate after 50 cycles or more, 100 cycles or more, or 150 cycles or more as compared to the same method performed without a single-stranded binding protein when both methods are used to sequence a plurality of the polynucleotide template. In some embodiments, the methods of the present disclosure that perform one or more steps in the presence of a single-stranded binding protein may have an error rate that is lower by 0.5% or greater, 1% or greater, 2% or greater, 3% or greater, 4% or greater, 5% or greater, 6% or greater, 7% or greater, 8% or greater, 9% or greater, 10% or greater, or 20% or greater as compared to same method performed without a single-stranded binding protein when both methods are used to sequence a plurality of the same polynucleotide.

In some embodiments, the method 100 may be a method for increasing the incorporation kinetics of a polynucleotide sequencing by synthesis process. Specifically, the method 100 that includes the use of a single-stranded binding protein may be a method of increasing the incorporation kinetics during sequencing of a polynucleotide template as compared to sequencing the same polynucleotide template using method 100 but without a single-stranded binding protein.

In some embodiments when the single-stranded binding protein is present during the incorporation step, the method has an increased rate of nucleotide incorporation (blocked, labeled nucleotide incorporation) into the copy polynucleotide strand as compared to the same method performed without the single-stranded binding protein when both methods are used to sequence a plurality of the same polynucleotide. In some embodiments when the single-stranded binding protein is present during the incorporation step, the method nucleotide incorporation rate that is 0.5% or greater faster, 1% or greater faster, 5% or greater faster, 10% or greater faster, 20% or greater faster, 30% or greater faster, 40% or greater faster, 50% or greater faster, or 60% or greater faster than the same method performed without the single-stranded binding protein when both methods are used to sequence a plurality of the same polynucleotide template.

### Kits and Cartridges

The present disclosure provides kits and cartridges for carrying out the methods of the present disclosure. The kits and cartridges are configured for use with a sequencing apparatus such as a sequencing instrument.

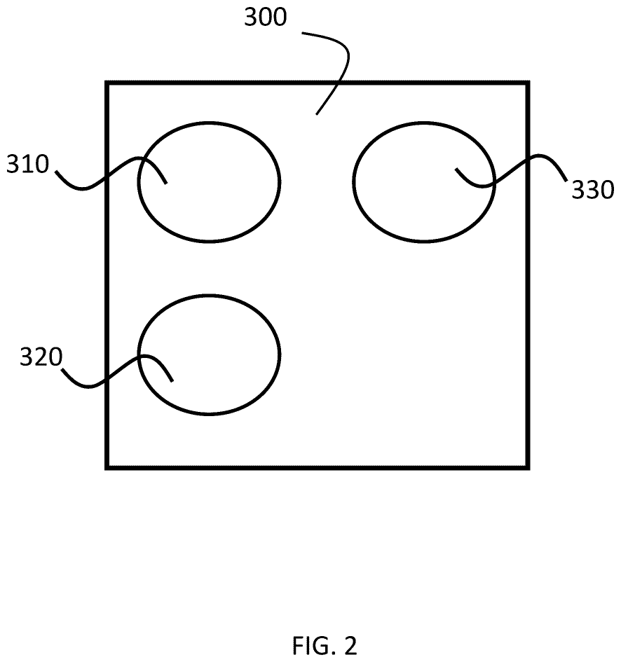

In some embodiments, cartridges for use with a sequencing apparatus may include a chamber from which a method composition (such as any method composition disclosed herein) may be withdrawn or expelled for use in any of the sequencing method steps (e.g., incorporation step, detection step, cleavage step, and washing step). For example, and with reference to FIG. 2, a cartridge 300 comprising a plurality of chambers 310, 320, 330 is shown. Each chamber 310, 320, and 330 contains a single method composition. A first method composition, for example, an incorporation composition (110C) for incorporating a blocked, labeled nucleotide into a copy polynucleotide strand complementary to at least a portion of a template polynucleotide strand (step 110), is disposed in the first chamber 310. A second method composition, for example, a detection composition (120C) for detecting the blocked, labeled nucleotide incorporated into the copy polynucleotide strand (step 120), is disposed in the second chamber 320. A third method composition, for example, a cleavage composition (130C) for removing a detectable label and a blocking group from the blocked, labeled nucleotide incorporated into the copy polynucleotide strand (step 130), is disposed in the third chamber 330. The cartridge may include one or more addition chambers. The additional chambers may contain a wash composition such as a post-incorporation wash composition (115C), a post-detection wash composition (125C), a post-cleavage wash composition (135C), or a universal wash buffer for completing one or more optional washing steps (steps 115, 125, 135).

Provided herein, are kits configured for use with the methods of the present disclosure. The kit may include one or more method compositions configured to perform one or more of the incorporation, detection, cleavage, and wash steps of method 100. A kit may be configured for use with a cartridge. For example, a kit may include the necessary compositions for disposing into the chambers of the cartridge.

The sequencing methods described herein may be performed in any suitable manner, using any suitable equipment. In some embodiments, the sequencing methods employ a solid support on which the multiple template polynucleotide strands are immobilized. The term immobilized as used herein is intended to encompass direct or indirect attachment to a solid support via covalent or non-covalent bond(s). In particular embodiments, all that is required is that the polynucleotides remain immobilized or attached to a support under conditions in which it is intended to use the support, for example in applications requiring nucleic acid amplification and/or sequencing. For example, oligonucleotides or primers may be immobilized such that a 3′ end is available for enzymatic extension and/or at least a portion of the sequence is capable of hybridizing to a complementary sequence. Immobilization can occur via hybridization to a surface attached primer, in which case the immobilized primer or oligonucleotide may be in 3′-5′ orientation. Alternatively, immobilization may occur by non-base-pairing hybridization, such as the covalent attachment.

By way of example, the polynucleotides may be attached to the surface by hybridization or annealing to one or more primers in a patch of primers. Hybridization may be accomplished, for example, by ligating an adapter to the ends of the template polynucleotides. The nucleic acid sequence of the adapter can be complementary to the nucleic acid sequence of the primer, thus, allowing the adapter to bind or hybridize to the primer on the surface. Optionally, the polynucleotides may be single- or double-stranded and adapters may be added to the 5′ and/or 3′ ends of the polynucleotides. Optionally, the polynucleotides may be double-stranded, and adapters may be ligated onto the 3′ ends of double-stranded polynucleotide. Optionally, polynucleotides may be used without any adapter. In some embodiments, template polynucleotides may be attached to a surface by interactions other than hybridization to a complementary primer. For example, a polynucleotide may be covalently attached to a surface using a chemical linkage such as those resulting from click chemistry or a receptor-ligand interaction such as streptavidin-biotin binding.

Primer oligonucleotides, oligonucleotide primers and primers are used throughout interchangeably and are polynucleotide sequences that are capable of annealing specifically to one or more polynucleotide templates to be amplified or sequenced. Generally, primer oligonucleotides are single-stranded or partially single-stranded. Primers may also contain a mixture of non-natural bases, non-nucleotide chemical modifications or non-natural backbone linkages so long as the non-natural entities do not interfere with the function of the primer. Optionally, a patch of primers on a surface of a solid support may comprise one or more different pluralities of primer molecules. By way of example, a patch may comprise a first, second, third, fourth, or more pluralities of primer molecules each plurality having a different sequence. It will be understood that for embodiments having different pluralities of primers in a single patch, the different pluralities of primers may share a common sequence so long as there is a sequence difference between at least a portion of the different pluralities. For example, a first plurality of primers may share a sequence with a second plurality of primers as long the primers in one plurality have a different sequence not found in the primers of the other plurality.

The template polynucleotides may be amplified on the surface of the solid support. Polynucleotide amplification includes the process of amplifying or increasing the numbers of a polynucleotide template and/or of a complement thereof that are present, by producing one or more copies of the template and/or or its complement. Amplification may be carried out by a variety of known methods under conditions including, but not limited to, thermocycling amplification or isothermal amplification. For example, methods for carrying out amplification are described in U.S. Publication No. 2009/0226975; WO 98/44151; WO 00/18957; WO 02/46456; WO 06/064199; and WO 07/010251; which are incorporated by reference herein in their entireties. Briefly, in the provided methods, amplification can occur on the surface to which the polynucleotide molecules are attached. This type of amplification can be referred to as solid phase amplification, which when used in reference to polynucleotides, refers to any polynucleotide amplification reaction carried out on or in association with a surface (e.g., a solid support). Typically, all or a portion of the amplified products are synthesized by extension of an immobilized primer. Solid phase amplification reactions are analogous to standard solution phase amplifications except that at least one of the amplification primers is immobilized on a surface (e.g., a solid support).

Suitable conditions include providing appropriate buffers/solutions for amplifying polynucleotides. Such solutions include, for example, an enzyme with polymerase activity, nucleotide triphosphates, and, optionally, additives such as DMSO or betaine. Optionally, amplification is carried out in the presence of a recombinase agent as described in U.S. Pat. No. 7,485,428, which is incorporated by reference herein in its entirety, which allows for amplification without thermal melting. Briefly, recombinase agents such as the RecA protein from E. coli (or a RecA relative from other phyla), in the presence of, for example, ATP, dATP, ddATP, UTP, or ATPγS, will form a nucleoprotein filament around single-stranded DNA (e.g., a primer). When this complex comes in contact with homologous sequences the recombinase agent will catalyze a strand invasion reaction and pairing of the primer with the homologous strand of the target DNA. The original pairing strand is displaced by strand invasion leaving a bubble of single-stranded DNA in the region, which serves as a template for amplification.

Solid-phase amplification may comprise a polynucleotide amplification reaction comprising only one species of oligonucleotide primer immobilized to a surface. Alternatively, the surface may comprise a plurality of first and second different immobilized oligonucleotide primer species. Solid phase nucleic acid amplification reactions generally comprise at least one of two different types of nucleic acid amplification, interfacial and surface (or bridge) amplification. For instance, in interfacial amplification the solid support comprises a template polynucleotide that is indirectly immobilized to the solid support by hybridization to an immobilized oligonucleotide primer, the immobilized primer may be extended in the course of a polymerase-catalyzed, template-directed elongation reaction (e.g., primer extension) to generate an immobilized polynucleotide that remains attached to the solid support. After the extension phase, the polynucleotides (e.g., template and its complementary product) are denatured such that the template polynucleotide is released into solution and made available for hybridization to another immobilized oligonucleotide primer. The template polynucleotide may be made available in 1, 2, 3, 4, 5 or more rounds of primer extension or may be washed out of the reaction after 1, 2, 3, 4, 5 or more rounds of primer extension.

In surface (or bridge) amplification, an immobilized polynucleotide hybridizes to an immobilized oligonucleotide primer. The 3′ end of the immobilized polynucleotide provides the template for a polymerase-catalyzed, template-directed elongation reaction (e.g., primer extension) extending from the immobilized oligonucleotide primer. The resulting double-stranded product “bridges” the two primers and both strands are covalently attached to the support. In the next cycle, following denaturation that yields a pair of single strands (the immobilized template and the extended-primer product) immobilized to the solid support, both immobilized strands can serve as templates for new primer extension.

Amplification may be used to produce colonies of immobilized polynucleotides. For example, the methods can produce clustered arrays of polynucleotide colonies, analogous to those described in U.S. Pat. No. 7,115,400; U.S. Publication No. 2005/0100900; WO 00/18957; and WO 98/44151, which are incorporated by reference herein in their entireties. “Clusters” and “colonies” are used interchangeably and refer to a plurality of copies of a polynucleotide having the same sequence and/or complements thereof attached to a surface. Typically, the cluster comprises a plurality of copies of a polynucleotide having the same sequence and/or complements thereof, attached via their 5′ termini to the surface. The copies polynucleotides making up the clusters may be in a single or double stranded form.

Thus, the plurality of template polynucleotides may be in a cluster, each cluster containing template polynucleotides of the same sequence. A plurality of clusters can be sequenced, each cluster comprising polynucleotides of the same sequence. Optionally, the sequence of the polynucleotides in a first cluster is different from the sequence of the nucleic acid molecules of a second cluster. Optionally, the cluster is formed by annealing to a primer on a solid surface a template polynucleotide and amplifying the template polynucleotide under conditions to form the cluster comprising the plurality of template polynucleotides of the same sequence. Amplification can be thermal or isothermal.

Each colony may comprise polynucleotides of the same sequences. In particular embodiments, the sequence of the polynucleotides of one colony is different from the sequence of the polynucleotides of another colony. Thus, each colony comprises polynucleotides having different nucleic acid sequences. All the immobilized polynucleotides in a colony are typically produced by amplification of the same polynucleotide. In some embodiments, it is possible that a colony of immobilized polynucleotides contains one or more primers without an immobilized polynucleotide to which another polynucleotide of different sequence may bind upon additional application of solutions containing free or unbound polynucleotides. However, due to the lack of sufficient numbers of free primers in a colony, this second or invading polynucleotide may not amplify to significant numbers. The second or invading polynucleotide typically is less than 1, 0.5, 0.25, 0.1, 0.001 or 0.0001% of the total population of polynucleotides in a single colony. Thus, the second or invading polynucleotide may not be optically detected or detection of the second or invading polynucleotide is considered background noise or does not interfere with detection of the original, immobilized polynucleotides in the colony. In such embodiments, the colony will be apparently homogeneous or uniform in accordance with the resolution of the methods or apparatus used to detect the colony.

The clusters may have different shapes, sizes and densities depending on the conditions used. For example, clusters may have a shape that is substantially round, multi-sided, donut-shaped or ring-shaped. The diameter or maximum cross section of a cluster may be from about 0.2 μm to about 6 μm, about 0.3 μm to about 4 μm, about 0.4 μm to about 3 μm, about 0.5 μm to about 2 μm, about 0.75 μm to about 1.5 μm, or any intervening diameter. Optionally, the diameter or maximum cross section of a cluster may be at least about 0.5 μm, at least about 1 μm, at least about 1.5 μm, at least about 2 μm, at least about 2.5 μm, at least about 3 μm, at least about 4 μm, at least about 5 μm, or at least about 6 μm. The diameter of a cluster may be influenced by a number of parameters including, but not limited to, the number of amplification cycles performed in producing the cluster, the length of the polynucleotide template, the GC content of the polynucleotide template, the shape of a patch to which the primers are attached, or the density of primers attached to the surface upon which clusters are formed. However, as discussed above, in all cases, the diameter of a cluster may be no larger than the patch upon which the cluster is formed. For example, if a patch is a bead, the cluster size will be no larger than the surface area of the bead. The density of clusters can be in the range of at least about 0.1/mm², at least about 1/mm², at least about 10/mm², at least about 100/mm², at least about 1,000/mm², at least about 10,000/mm² to at least about 100,000/mm². Optionally, the clusters have a density of, for example, 100,000/mm² to 1,000,000/mm² or 1,000,000/mm² to 10,000,000/mm². The methods provided herein can produce colonies that are of approximately equal size. This occurs regardless of the differences in efficiencies of amplification of the polynucleotides of different sequence.

Clusters may be detected, for example, using a suitable imaging means, such as, a confocal imaging device or a charge coupled device (CCD) or CMOS camera. Exemplary imaging devices include, but are not limited to, those described in U.S. Pat. Nos. 7,329,860; 5,754,291; and 5,981,956; and WO 2007/123744, each of which is herein incorporated by reference in its entirety. The imaging apparatus may be used to determine a reference position in a cluster or in a plurality of clusters on the surface, such as the location, boundary, diameter, area, shape, overlap and/or center of one or a plurality of clusters (and/or of a detectable signal originating therefrom). Such a reference position may be recorded, documented, annotated, converted into an interpretable signal, or the like, to yield meaningful information.

As used herein the term support refers to a substrate for attaching polynucleotides. A support is a material having a rigid or semi-rigid surface to which a polynucleotide can be attached or upon which nucleic acids can be synthesized and/or modified. Supports can include any resin, gel, bead, well, column, chip, flowcell, membrane, matrix, plate, filter, glass, controlled pore glass (CPG), polymer support, membrane, paper, plastic, plastic tube or tablet, plastic bead, glass bead, slide, ceramic, silicon chip, multi-well plate, nylon membrane, fiber optic, and PVDF membrane.

A support may include any flat wafer-like substrates and flat substrates having wells, such as a microtiter plate, including 96-well plates. Exemplary flat substrates include chips, slides, etched substrates, microtiter plates, and flow cell reactors, including multi-lane flow cell reactors having multiple microfluidic channels, such as the eight-channel flow cell used in the cBot sequencing workstation (Illumina, Inc., San Diego, CA). Exemplary flow cells are described in WO 2007/123744, which is incorporated herein by reference in its entirety. Optionally, the flowcell is a patterned flowcell. Suitable patterned flowcells include, but are not limited to, flowcells described in WO 2008/157640, which is incorporated by reference herein in its entirety.

A support may also include beads, including magnetic beads, hollow beads, and solid beads. Beads may be used in conjunction with flat supports, such flat supports optionally also containing wells. Beads, or alternatively microspheres, refer generally to a small body made of a rigid or semi-rigid material. The body may have a shape characterized, for example, as a sphere, oval, microsphere, or other recognized particle shape whether having regular or irregular dimensions. The sizes of beads, in particular, include, without limitation, about 1 μm, about 2 μm, about 3 μm, about 5 μm, about 10 μm, about 20 μm, about 30 μm, about 40 μm, about 60 μm, about 100 μm, about 150 μm or about 200 μm in diameter. Other particles may be used in ways similar to those described herein for beads and microspheres.

The composition of a support may vary depending, for example, on the format, chemistry and/or method of attachment and/or on the method of nucleic acid synthesis. Support materials that can be used in accordance with the present disclosure include, but are not limited to, polypropylene, polyethylene, polybutylene, polyurethanes, nylon, metals, and other suitable materials. Exemplary compositions include supports, and chemical functionalities imparted thereto, used in polypeptide, polynucleotide and/or organic moiety synthesis. Such compositions include, for example, plastics, ceramics, glass, polystyrene, melamine, methylstyrene, acrylic polymers, paramagnetic materials, thoria sol, carbon graphite, titanium dioxide, latex or cross-linked dextrans such as Sepharose™, cellulose, nylon, cross-linked micelles and Teflon™, as well as any other materials which can be found described in, for example, “Microsphere Detection Guide” from Bangs Laboratories, Fishers IN, which is incorporated herein by reference. A support particle may be made of cross-linked starch, dextrans, cellulose, proteins, organic polymers including styrene polymers including polystyrene and methylstyrene as well as other styrene co-polymers, plastics, glass, ceramics, acrylic polymers, magnetically responsive materials, colloids, thoriasol, carbon graphite, titanium dioxide, nylon, latex, or TEFLON®. “Microsphere Detection Guide” from Bangs Laboratories, Fishers, Inc., hereby incorporated by reference in its entirety, is a helpful guide. Further exemplary supports within the scope of the present disclosure include, for example, those described in US Application Publication No. 02/0102578 and U.S. Pat. No. 6,429,027, both of which are incorporated herein by reference in their entirety.

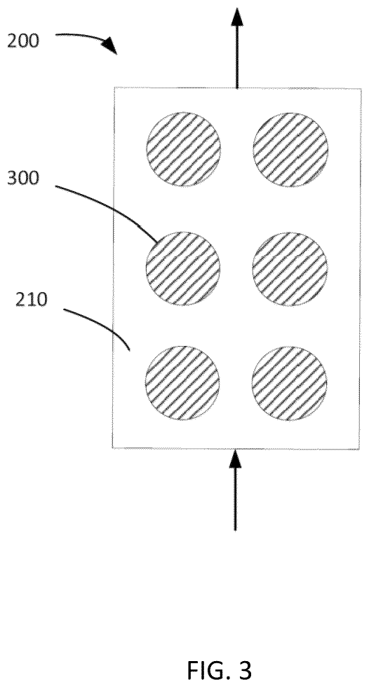

For example, and with reference to FIG. 3, an embodiment of a solid support 200, such as a flow cell, is shown. The solid support 200 has a surface 210 to which clusters 300 containing multiple template polynucleotide strands having the same nucleotide sequence are bound to the surface 210 of the solid support 200. The surface 210 of the solid support 200 may be planar.

Fluid compositions containing reagents, wash buffers, and the like (e.g., method compositors) may flow over the surface 210 of the solid support 200 to interact with the template polynucleotides in the clusters 300. The flow of the compositions may occur in any direction, such as the direction indicated by the arrows in FIG. 3.

Sequencing apparatus with which the flow cell 200 may be used may be configured to flow reagents and compositions across the surface 210 to interact with the template strands in the clusters 300. For example, the apparatus may cause method compositions to flow across the surface 210 of the solid support 200, such as a flow cell, to interact with the template polynucleotides in the clusters 300 at the appropriate times to carry out sequencing of the polynucleotide template strands.

Each cluster 300 may contain the same template polynucleotides or different polynucleotides than another cluster 300.

The template polynucleotides to be sequenced may be obtained from any biological sample using known, routine methods. Suitable biological samples include, but are not limited to, a blood sample, biopsy specimen, tissue explant, organ culture, biological fluid or any other tissue or cell preparation, or fraction or derivative thereof or isolated therefrom. The biological sample can be a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, stem cells, germ cells (e.g. sperm, oocytes), transformed cell lines and the like. For example, polynucleotide molecules may be obtained from primary cells, cell lines, freshly isolated cells or tissues, frozen cells or tissues, paraffin embedded cells or tissues, fixed cells or tissues, and/or laser dissected cells or tissues. Biological samples can be obtained from any subject or biological source including, for example, human or non-human animals, including mammals and non-mammals, vertebrates and invertebrates, and may also be any multicellular organism or single-celled organism such as a eukaryotic (including plants and algae) or prokaryotic organism, archaeon, microorganisms (e.g. bacteria, archaea, fungi, protists, viruses), and aquatic plankton.

Once the polynucleotides are obtained, a plurality of polynucleotides molecules of different sequence for use in the provided methods may be prepared using a variety of standard techniques available and known. Exemplary methods of polynucleotide molecule preparation include, but are not limited to, those described in Bentley et al., Nature 456:49-51 (2008); U.S. Pat. No. 7,115,400; and U.S. Patent Application Publication Nos. 2007/0128624; 2009/0226975; 2005/0100900; 2005/0059048; 2007/0110638; and 2007/0128624, each of which is herein incorporated by reference in its entirety. The template polynucleotides may contain a variety of sequences including, but not limited to, universal sequences and known or unknown sequences. For example, polynucleotide may comprise one or more regions of known sequence (e.g., an adaptor) located on 5′ and/or 3′ ends. Such template polynucleotides may be formed by attaching adapters to the ends of a polynucleotides of unknown sequence. When the polynucleotides comprise known sequences on 5′ and 3′ ends, the known sequences may be the same or different sequences. Optionally, a known sequence located on 5′ and/or 3′ ends of the polynucleotides is capable of hybridizing to one or more primers immobilized on the surface. For example, a polynucleotide comprising a 5′ known sequence may hybridize to a first plurality of primers while 3′ known sequence may hybridize to a second plurality of primers. Optionally, polynucleotides comprise one or more detectable labels. The one or more detectable labels may be attached to the polynucleotide template at the 5′ end, at the 3′ end, and/or at any nucleotide position within the polynucleotide molecule. The polynucleotides for use in the provided methods may comprise the polynucleotide to be amplified and/or sequenced and, optionally, short nucleic acid sequences at 5′ and/or 3′ end(s).

A short nucleic acid sequence that is added to the 5′ and/or 3′ end of a polynucleotide may be a universal sequence. A universal sequence is a region of nucleotide sequence that is common to, i.e., shared by, two or more polynucleotides, where the two or more polynucleotides also have regions of sequence differences. A universal sequence that may be present in different members of a plurality of polynucleotides may allow the replication or amplification of multiple different sequences using a single universal primer that is complementary to the universal sequence. Similarly, at least one, two (e.g., a pair) or more universal sequences that may be present in different members of a collection of polynucleotides may allow the replication or amplification of multiple different sequences using at least one, two (e.g., a pair) or more single universal primers that are complementary to the universal sequences. Thus, a universal primer includes a sequence that may hybridize specifically to such a universal sequence. The polynucleotide may be modified to attach universal adapters (e.g., non-target nucleic acid sequences) to one or both ends of the different target sequences, the adapters providing sites for hybridization of universal primers. This approach has the advantage that it is not necessary to design a specific pair of primers for each polynucleotide to be generated, amplified, sequenced, and/or otherwise analyzed; a single pair of primers can be used for amplification of different polynucleotides provided that each polynucleotide is modified by addition of the same universal primer-binding sequences to its 5′ and 3′ ends.

The polynucleotides may also be modified to include any nucleic acid sequence desirable using standard, known methods. Such additional sequences may include, for example, restriction enzyme sites, or indexing tags in order to permit identification of amplification products of a given nucleic acid sequence.

As used herein, the term different when used in reference to two or more polynucleotides means that the two or more polynucleotides have nucleotide sequences that are not the same. For example, two polynucleotides can differ in the content and order of nucleotides in the sequence of one polynucleotide compared to the other polynucleotide. The term can be used to describe polynucleotides whether they are referred to as copies, amplicons, templates, targets, primers, oligonucleotides, or the like.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation may not be explicitly disclosed, each is specifically contemplated and described herein. For example, if a method is disclosed and discussed and a number of modifications that can be made to the method steps are discussed, each and every combination and permutation of the method steps, and the modifications that are possible are specifically contemplated unless specifically indicated to the contrary. Likewise, any subset or combination of these is also specifically contemplated and disclosed. This concept applies to all aspects of this disclosure. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

### EXAMPLES

The sequence of polymerase Pol(X) used in the examples can be found as SEQ ID NO: 5 in U.S. application Ser. No. 18/373,620 filed Sep. 27, 2023, which is hereby incorporated by reference in its entirety. The sequence of polymerase Pol (Y) can be found in U.S. Pat. No. 11,001,816, (filed Dec. 4, 2019, issued May 11, 2021) and U.S. Pat. No. 11,634,697 (filed Apr. 9, 2021, issued Apr. 25, 2023), both of which are hereby incorporated by reference in its entirety. The SSBs used in the Examples include Rev101-F255P (SEQ ID NO: 2B), Rev 1010 (SEQ ID NO: 1B), Rev 94 (SEQ ID NO: 3B), and Rev 93 (SEQ ID NO: 4B).

### Example 1

### Materials and Methods:

General protocol for the sequencing methods used in the examples.

For sequencing the library was loaded onto the sequencer using standard library denaturation and dilution conditions. Next, patterned flowcell Exclusion Amplification (ExAmp) was used to make clusters. After cluster formation, an exonuclease was used to remove excess surface primers. Next, the CCL1, chemical linearization reagent 1 was used to linearise surface primers. Sequencing by synthesis was the conducted using the standard protocol and reagents from Illumina Inc. Specifically the sequencing was conducted using the following steps:
1) incorporation of a blocked, labeled nucleotide using the standard incorporation mix (e.g., incorporation composition) from Illumina Inc;
2) washing using the standard washing reagent mixture (e.g., post-incorporation composition) form Illumina Inc;
3) detecting the identity of the blocked, labelled nucleotide using the standard scan mix (e.g., detection composition) from Illumina Inc;
4) deblocking the blocked, labeled nucleotide using the standard deblocking reagent and scavenger containing wash mixture (e.g., cleavage composition) from Illumina Inc;
5) washing using the standard washing mix (e.g., post-cleavage wash composition) from Illumina Inc; and
6) repeating steps 1 to 5.

The washing reagent mixture of step 2 and the washing reagent mixture of step 5 are the same.

### Example 1A: Assessing the Impact of the Inclusion of a Single-Stranded Binding Protein within the Incorporation Composition and Wash Composition on Error Rate

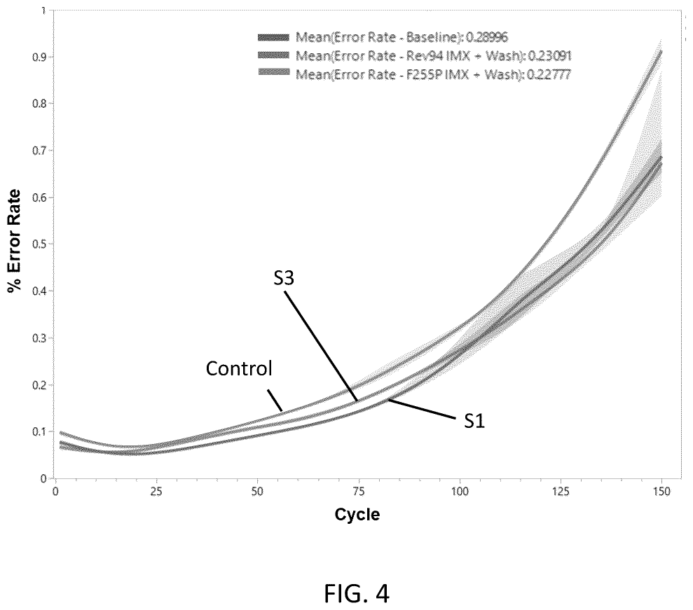

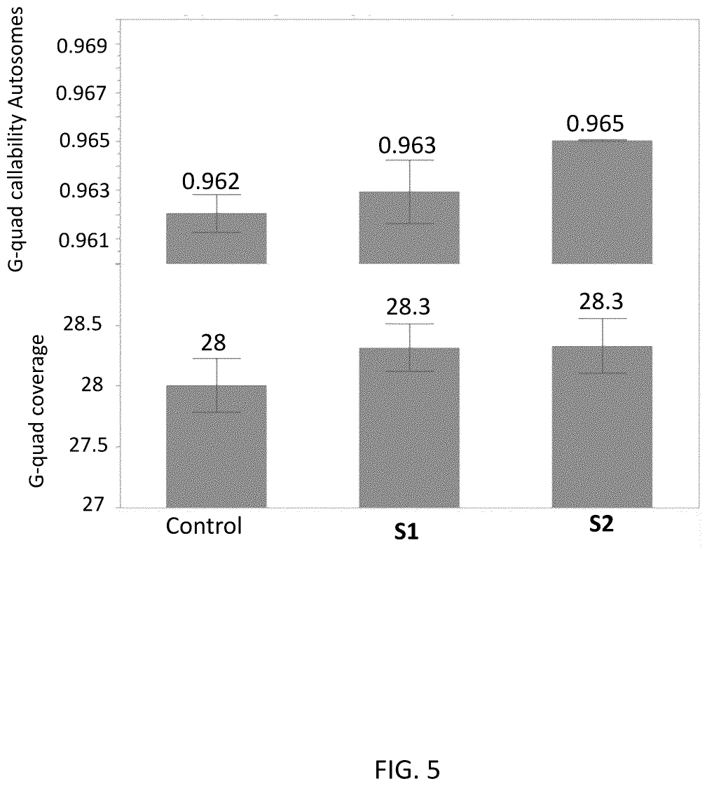

Protocol used to generate the data in FIG. 4 and FIG. 5.

SSB were tested within the incorporation mix on NextSeq2k with a P2 cartridge (Illumina Inc.) and flow cell modified to run X-Leap Chemistry according to Illumina Inc. protocol (Illumina, Inc.). For this, the standard Illumina Inc., incorporation mix well was replaced with incorporation mix containing X-Leap blocked ffNs and Pol(X). The SSB was include either only in the incorporation mix, the detection composition, or in both the incorporation mix and the standard washing mixture used in step 2 and 5 of the General Sequencing Protocol. The samples tested in Example 1A are shown in Table 2.

TABLE 2
Samples Tested in Example 1B

| | Incorporation | Wash composition | Detection |
|---|---|---|---|
| Sample | composition | (steps 2 and 5) | composition |
| control | | | |
| S1 | 0.2 mg/ml Rev101- | 0.2 mg/ml Rev101- | |
| | F225P | F225P | |
| S2 | | | 0.5 mg/ml |
| | | | Rev101-F225P |
| S3 | 0.2 mg/ml Rev94 | 0.2 mg/ml Rev94 | |

The inclusion of an SSBs (Rev94 or Rev 101 F225P) to both the incorporation composition and wash composition at a concentration of 0.2 mg/ml appears to cause decrease in error rate as compared to completing sequencing without an SSB in the incorporation composition and wash composition (FIG. 4).

G-quadruplex callability indicates how well the method was able to sequence through a series of known G-quadruplex sequences in the template polynucleotide. The higher the callability the more nucleotides within the known G-quadruplex sequence were correctly identified.

G-quad coverage is the amount of nucleotides (reads) that span a G-quadruplex region of the polynucleotide template. To determine G-quad coverage, the reads in the G-quadruplex region were randomly downsampled to 30 reads per position as a representative of the sequencing quality at each position. An increase in G-quadruplex coverage indicates an increase in usable data per position.

Analysis of the sequencing scan metrics of S1 and S3 revealed that the inclusion of SSBs in the incorporation composition (IMX) and wash composition (wash) (S1), or in the detection composition (S2) at a higher concentration, improves G-quadruplex coverage and callability (FIG. 5).

### Example 1B: Assessing the Impact of the Inclusion of a Single-Stranded Binding Protein and/or Lithium Chloride within the Incorporation Composition and Wash Composition on Q-Quadruplex Resolution During Sequencing

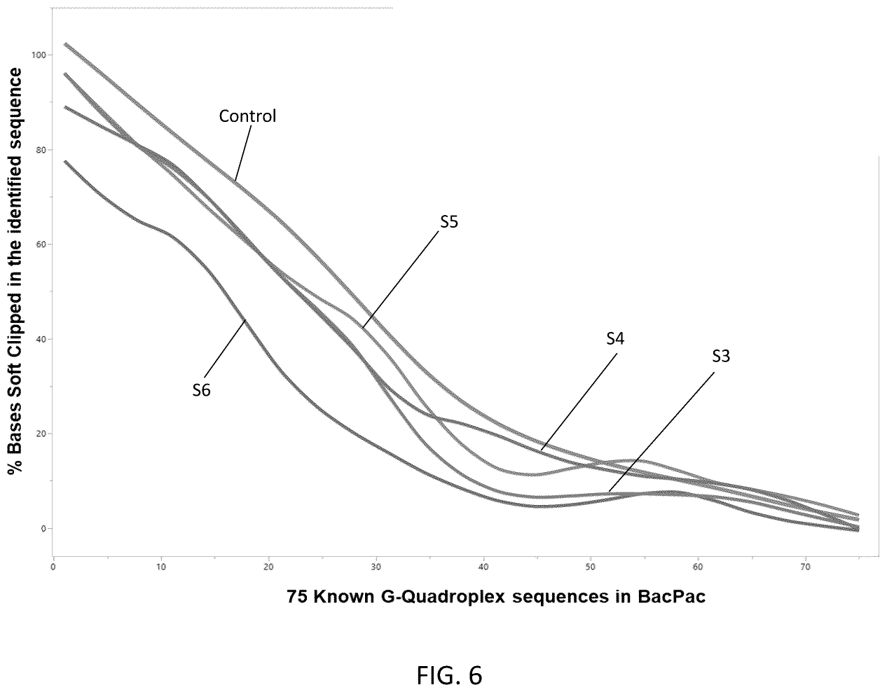

Protocol used to generate the data in FIG. 6.

SSBs were tested in conjunction with modified salt compositions of the incorporation mixture (used in step 1 of the General Sequencing Protocol). Table 3 shows the samples tested in Example 1B. In Table 3, where no modification is indicated, the composition used was the standard composition as described in the General Sequencing Protocol. The standard storage buffer for Pol(X) included potassium ions. Since Pol(X) is included in the incorporation mixture, the standard incorporation mixture includes potassium ions. For sample S3, the incorporation composition contained no potassium ions. The potassium ions were removed from the Pol(X) polymerase storage buffer. In this Example, the polymerase stored in the presence of potassium ions is termed Pol(X)a and the polymerase stored free of potassium ion is termed Pol(X)b. Sample S3 still included—sodium ions (from sodium chloride and sodium glycine). For sample S4, the sodium chloride in the incorporation mix buffer was replaced by lithium chloride. Sample S4 included potassium ions from the Pol(X)a storage buffer. In sample S6, all of the treatments in samples S3 to S5 were combined. Specifically, the incorporation mixture included an SSB, LiCl in place of sodium chloride in the incorporation buffer, and no potassium (Pol(X)b used).

Table 3 shows the components of the incorporation composition, wash composition, and detection composition of each sample tested that deviate from the standard composition components.

TABLE 3
Samples Tested in Example 1B

| | | Poly- | Wash | Detection | Mean % of |
|---|---|---|---|---|---|
| | Incorporation | merase | com- | com- | bases soft- |
| Sample | composition | used | position | position | clipped |
| control | | Pol(X)a | | | 39.1% |
| S3 | no potassium | Pol(X)b | | | 31.0% |
| S4 | LiCl | Pol(X)a | | | 33.8% |
| S5 | 0.2 mg/ml | Pol(X)a | | | 34.5% |
| | Rev101-F225P | | | | |
| S6 | no potassium, | Pol(X)b | | | 22.7% |
| | LiCl, 0.2 mg/ml | | | | |
| | Rev101-F225P | | | | |

FIG. 6 displays the average soft-clipping values for samples S3-S6 of Table 3. The mean percent of bases softclipped for each sample is also shown in Table 3. When a series of bases are miscalled (errors) the read and bases are consequently soft clipped (essentially removed). Percent soft-clipping of bases is a measure of the percent of bases that were removed due to an error. Lower percent soft clip values indicate a higher proportion of bases have been called correctly. When an SSB is included in the incorporation composition (S5) a shift in the levels of softclipping is observed. Additionally, the removal of potassium ions from the incorporation composition (S3) and inclusion of lithium chloride in the incorporation composition (S4) resulted in a decrease in softclipping. When the SSBs are used in combination with potassium ion removal and sodium chloride replacement with LiCl (S6), the levels of softclipping decrease further (Table 3 and FIG. 6).

### Example 1C: Assessing the Impact of the Inclusion of a Single-Stranded Binding Protein in the Detection Composition on Sequencing with Different Laser Profiles

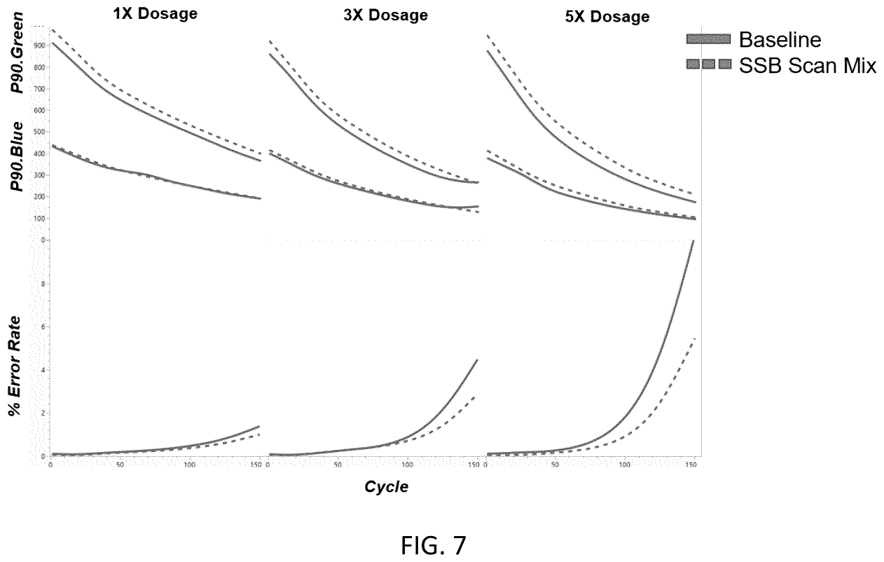

Protocol used to generate the data in FIG. 7.

Rev 101 F225P (0.5 mg/ml) was also tested in the standard scan mix reagent (used during step 3 of the General Sequencing Protocol), both with standard laser power profiles and with a laser dosage titration from OX to 10× standard exposure levels (3× power and 5× power). This was done to assess if the inclusion of an SSB in the scan mix (e.g., detection composition) is able to provide a benefit when polynucleotide templates are sequenced with higher power Blue and Green lasers. For this experiment the laser exposure times were changed on the instruments imaging software such that specific regions of the flow cell used for each experiment would receive a different amount/time of laser exposure corresponding to a time either 0 to 10 times (1×, 3×, 5×) that of the standard exposure time for this platform at any given cycle.

At 1× Laser power the percent error rate when including the SSB in the detection composition is similar to the control (no SSB included in detection composition; FIG. 7). However, when the laser exposure time is elevated to 3X and 5X, the presence of the SSB appears to cause a dose dependent decrease in error rate as compared to the detection composition devoid of the SSB (FIG. 7).

### Example 1D: Assessing the Impact of the Inclusion of a Single-Stranded Binding Protein in the Incorporation Composition on Incorporation Kinetics

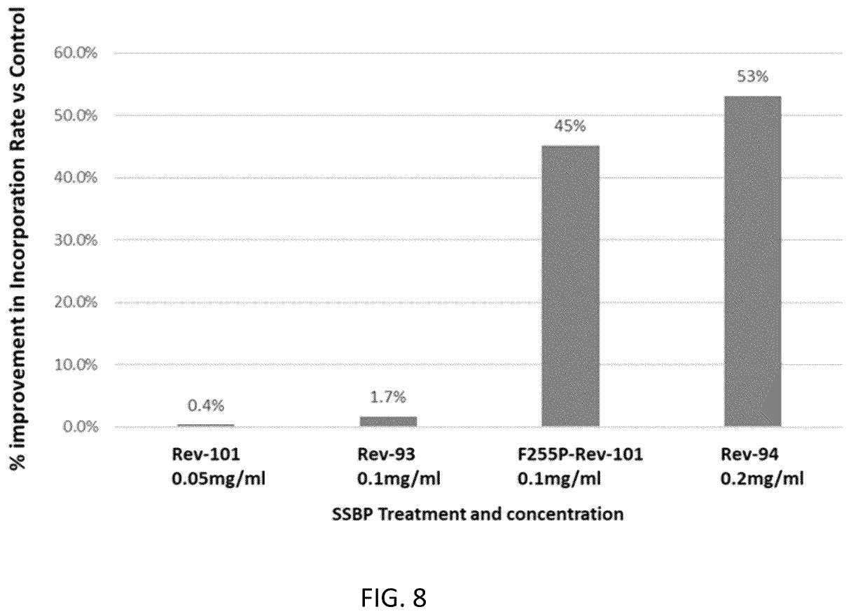

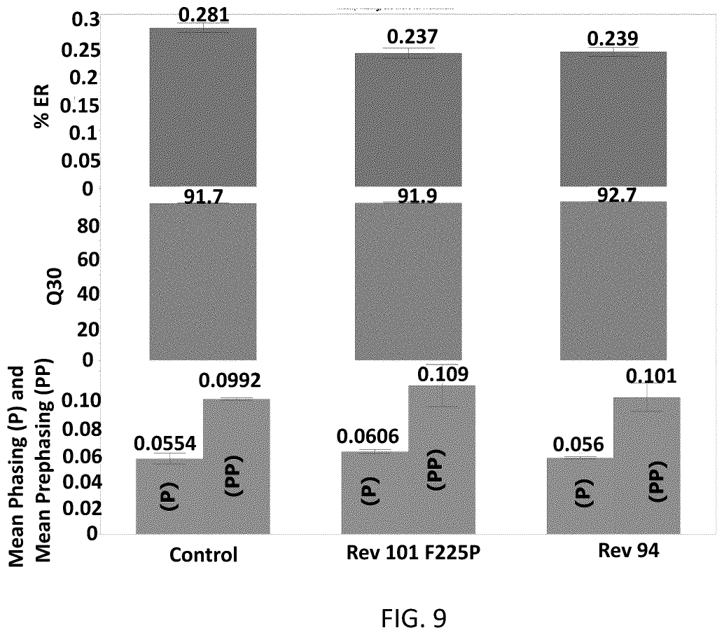

Protocol used to generate the data in FIG. 8 and FIG. 9.

The sequence protocol to generate the data in FIG. 8 and FIG. 9 followed the General Sequencing Protocol where either 0.05 mg/ml Rev 101, 0.1 mg/ml Rev 93, 0.1 mg/ml F225P-Rev101, or 0.2 mg/ml Rev-94 were added to the incorporation mix (step 1 in the General Sequencing Protocol).

FIG. 8 summarizes the percent incorporation improvement when adding SSBs into the incorporation mix. This was tested by a standard kinetics assay. The assay works using mono polynucleotide templates and performs a series of kinetic cycles. The incorporation mix were kinetic tested over a series of time points and percent incorporation is measured at each time using fluorescence detection. A percent incorporation vs. time graph was generated for multiple incorporation mixture concentrations. This data was used to calculate key kinetic parameters including Vmax and Km. The data in FIG. 8 summarizes the improvement to these kinetic parameters for each of the SSBs against a control value. This experiment was conducted a while ago using Pol (Y).

Shown on FIG. 9 are the standard primary metrics measured in sequencing. On addition of two different SSBs (F225P and Rev94 at 0.2 mg/ml) there is an improvement to error rate while causing no other impact to other key sequencing metrics e.g. Q30, phasing and prephasing.

### Example 1E: Assessing the Impact of the Inclusion of a Single-Stranded Binding Protein in Sequencing Step on G-Quadruplex Resolution

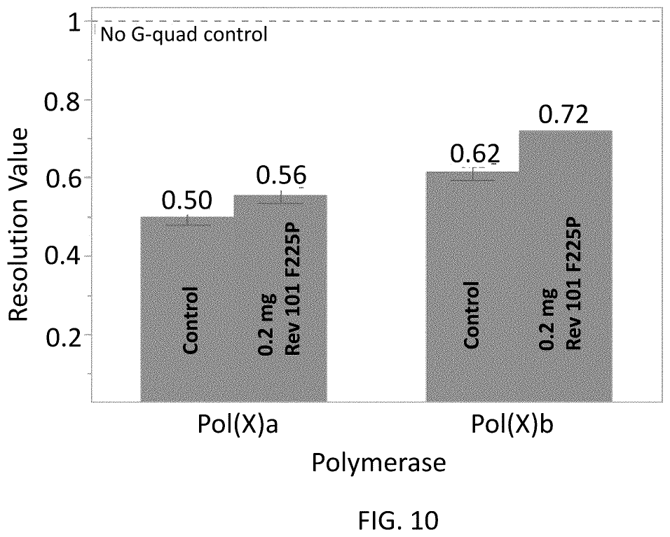

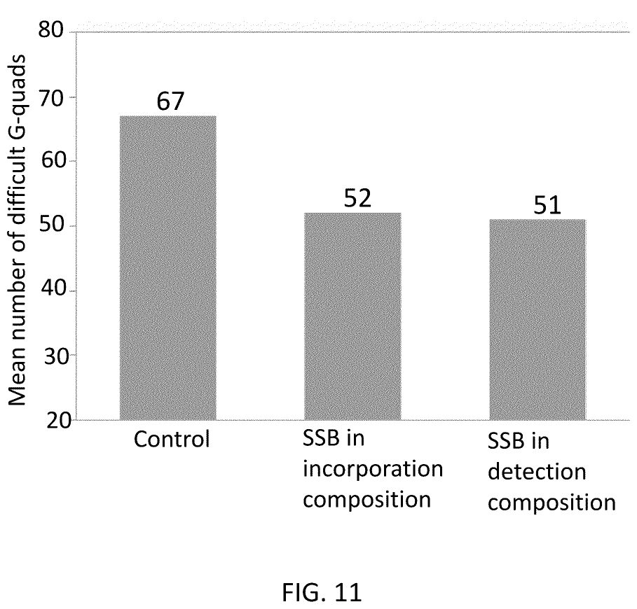

Protocol used to generate the data in FIG. 10 and FIG. 11.

The sequence protocol to generate the data in FIG. 10 and FIG. 11 followed the General Sequencing Protocol.

For FIG. 10, Rev101 F225P (0.2 mg/ml) was added to the incorporation mixture. The incorporation mixture included either Pol(X)a (inclusion of potassium ions in the polymerase storage buffer) or Pol(X)b (no potassium ions in the polymerase storage buffer). The template polynucleotide includes the sequence TTGGGGGAGGGGGAGGGGGAGGGG (SEQ ID NO: 13), a sequence known to form G-quadruplexes. The resolution value is calculated as the difference between the expected and detected intensity traces. It is used to quantify the loss of signal associated with the presence of secondary structures (G-Quads). A resolution value of 1 indicates the expected intensity matches the detected intensity and is indicative of the presence of no secondary structures for G-Quadruplexes. The inclusion of an SSB in the incorporation mix with either Pol(X)a or Pol(X)b resulted in an improvement to the resolution value.

For FIG. 11, Rev101 F225P was added to either the scan mix (step 3 in General Sequencing Protocol) or the incorporation mix. The polynucleotide template sequenced included 519 G-quadruplexes. Not all of the G-quadruplexes in this sequence cause issues for sequencing. FIG. 11 shows the number of difficult G-Quads in BacPack. Difficult G-Quads are defined by the following metrics: Softclipping >10%, MapQ>40, Normalized coverage >0.2. On addition of SSBs in either the incorporation mix or the scan mix, a decrease in the number of difficult G-Quads was observed.

### Example 1F: Assessing the Binding Affinity of TthF225P SSB and Tth SSB

An electrophoretic mobility shift assay was used to determine the binding affinity (Kd) for the Tth F255P SSB and Tth SSB. An oligonucleotide of 35 bases labelled at the 5′ end with fluorescein (FAM) dye was used as the substrate. The sequence of the oligonucleotide is 5′ FAM GTG TAG ATC TCG GTG GTC GCC GTA TCA TTA AAA AA (SEQ ID NO: 14). A titration of Tth F255P SSB or Tth SSB was used ranging from 0.1 to 4 μM. The reaction buffer was 50 mM Tris pH 7.5, 50 mM NaCl, and 0.1 mM DTT with each reaction utilizing 0.25 nm of labelled oligonucleotide. The reactions were incubated at 60° C. for 10 minutes. The reactions were resolved on 10% acrylamide native TBE gel and imaged with Typhoon Scanner. The fraction of bound versus unbound for each concentration tested was determined and fitted for Kd analysis. FIG. 12 shows the binding isotherms and Kds.

### OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment, any portion of the embodiment, or in combination with any other embodiments or any portion thereof.

As is set forth herein, it will be appreciated that the disclosure comprises specific embodiments and examples of base editing systems to effect a nucleobase alteration in a gene and methods of using same for treatment of disease including compositions that comprise such base editing systems, designs and modifications thereto; and specific examples and embodiments describing the synthesis, manufacture, use, and efficacy of the foregoing individually and in combination including as pharmaceutical compositions for treating disease and for in vivo and in vitro delivery of active agents to mammalian cells under described conditions.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:

Following is an example of enumerated clauses:
1. A polynucleotide sequencing method comprising:
   (a) incorporating a blocked, labeled nucleotide into a copy polynucleotide strand that is complementary to and hybridized with at least a portion of a template polynucleotide strand in a sequencing complex;
   (b) detecting the identity of the blocked, labeled nucleotide;
   (c) chemically removing a label and blocking moiety from the blocked, labeled nucleotide incorporated into the copy strand;
   (d) repeating steps (a)-(c),
   wherein at least one of steps (a)-(c) are performed in the presence of a single-stranded binding (SSB) protein.
2. A method for improving secondary structure resolution of a polynucleotide sequencing by synthesis process, the method comprising:
   (a) incorporating a blocked, labeled nucleotide into a copy polynucleotide strand that is complementary to and hybridized at least a portion of a template polynucleotide strand in a sequencing complex;
   (b) detecting the identity of the blocked, labeled nucleotide;
   (c) chemically removing a label and blocking moiety from the blocked, labeled nucleotide incorporated into the copy strand;
   (d) repeating steps (a)-(c),
   wherein at least one of steps (a)-(c) are performed in the presence of a single-stranded binding (SSB) protein.
3. The method of clause 2, wherein the method is for improving G-quadruplex resolution of a sequencing by synthesis process.
4. The method of clause 2 or 3, wherein the method comprises:
   repeating steps (a)-(c) to sequence at least a portion of a plurality of polynucleotide templates having the same sequence;
   wherein completion of sequencing the at least a portion of a plurality of polynucleotides results in a resolution value (a percent of nucleotides correctly identified in one or more regions of known secondary structure in the sequenced plurality of polynucleotide templates) that is larger than a resolution value of the same method completed in the absence a single-stranded binding protein.
5. The method of clause 4, wherein the resolution value is 5% or greater than the resolution value of the same method completed without a single-stranded binding protein.
6. The method of clause 5, wherein the resolution value is 10% or greater than the resolution value of the same method completed without a single-stranded binding protein.
7. The method of any one of clauses 2 through 6, wherein step (a) is performed in the presence of the single-stranded binding protein.
8. A method for lowering an error rate of a polynucleotide sequencing by synthesis process, the method comprising:
   (a) incorporating a blocked, labeled nucleotide into a copy polynucleotide strand that is complementary to and hybridized with at least a portion of a template polynucleotide strand in a sequencing complex;
   (b) detecting the identity of the blocked, labeled nucleotide;
   (c) chemically removing a label and blocking moiety from the blocked, labeled nucleotide incorporated into the copy strand;
   (d) repeating steps (a)-(c),
   wherein at least one of steps (a)-(c) are performed in the presence of a single-stranded binding (SSB) protein.
9. The method of clause 8, wherein the method comprises:
   repeating steps (a)-(c) at least 50 times to sequence at least a portion of a plurality of polynucleotide templates having the same sequence;
   wherein completion of sequencing the at least a portion of a plurality of polynucleotides results in an error rate (a percent of nucleotides correctly identified in one or more regions of known secondary structure in the sequenced plurality of polynucleotide templates) that is lower than an error rate of the same method completed without a single-stranded binding protein.
10. The method of clause 9, wherein the error rate is 5% or greater lower than the error rate of the same method completed without a single-stranded binding protein.
11. The method of clause 8, wherein the resolution value is 10% or greater lower than the error rate of the same method completed without a single-stranded binding protein.
12. A method for increasing incorporation kinetics of a polynucleotide sequencing by synthesis process, the method comprising:
   (a) incorporating a blocked, labeled nucleotide into a copy polynucleotide strand that is complementary to and hybridized with at least a portion of a template polynucleotide strand in a sequencing complex, wherein step (a) is performed in the presence of a single-stranded binding (SSB) protein;
   (b) detecting the identity of the blocked, labeled nucleotide;
   (c) chemically removing a label and blocking moiety from the blocked, labeled nucleotide incorporated into the copy strand;
   (d) repeating steps (a)-(c).
13. The method of clause 12, wherein completion of step (a) results in an incorporation rate, and wherein the incorporation rate is faster than an incorporation rate of performing step (a) without the single-stranded binding protein.
14. The method of clause 13, wherein the incorporation rate is 1% or greater faster than an incorporation rate of performing step (a) without the single-stranded binding protein.
15. The method of clause 13, wherein the incorporation rate is 10% or greater faster than an incorporation rate of performing step (a) without the single-stranded binding protein.
16. The method of any one of clauses 1 to 15, wherein:
   step (a) further comprises exposing the sequencing complex to an incorporation composition;
   step (b) further comprises exposing the sequencing complex to a detection composition;
   step (c) further comprises exposing the sequencing complex to a cleavage composition; and
   wherein at least one of the incorporation composition, the detection composition, and cleavage composition comprise the single-stranded binding protein.
17. The method of clause 16, wherein the single-stranded binging protein is present at a concentration of 0.01 mg/ml to 2 mg/ml.
18. The method of clause 16 or clause 17, wherein the incorporation composition comprises the single-stranded binding protein.
19. The method of any one of clauses 16 to 18, wherein the detection composition comprises the single-stranded binding protein.
20. The method of any one of clauses 16 to 19, wherein the cleavage composition comprises the single-stranded binding protein.
21. The method of any one of clauses 1 to 20, wherein the method further comprises washing the sequence complex with a wash composition.
22. The method of clause 21, wherein the wash composition comprises the single-stranded binding protein.
23. The method of any one of clauses 1 to 22, wherein at least one of the steps (a)-(c) is performed in the absence of potassium ions.
24. A cartridge for use with a sequencing apparatus, the cartridge comprising:
   a first chamber having an incorporation composition for incorporating a blocked, labeled nucleotide into a copy polynucleotide strand complementary to and hybridized with at least a portion of a template polynucleotide strand disposed within;
   a second chamber having a detection composition for detecting the identity of the blocked, labeled nucleotide disposed within; and
   a third chamber having a cleavage composition for chemically removing a label and blocking moiety from the blocked, labeled nucleotide incorporated into the copy strand disposed within;
   wherein at least one of the incorporation composition, the detection composition, and the cleavage composition comprises a single-stranded binding protein.
25. The cartridge of clause 24 further comprising a fourth chamber having a wash composition disposed within.
26. The cartridge of clause 25, where the wash composition comprises a single-stranded binding protein.
27. A kit for use with a sequencing apparatus, the kit comprising:
   a first chamber having an incorporation composition for incorporating a blocked, labeled nucleotide into a copy polynucleotide strand complementary to and hybridized with at least a portion of a template polynucleotide strand disposed within;
   a second chamber having a detection composition for detecting the identity of the blocked, labeled nucleotide disposed within; and
   a third chamber having a cleavage composition for chemically removing a label and blocking moiety from the blocked, labeled nucleotide incorporated into the copy strand disposed within;
   wherein at least one of the incorporation composition, the detection composition, and the cleavage composition comprises a single-stranded binding protein.
28. The kit of clause 27, further comprising a fourth chamber having a wash composition disposed within.
29. The cartridge of clause 28, where the wash composition comprises a single-stranded binding protein.
30. A polynucleotide sequencing method comprising:
   (a) incorporating a blocked, labeled nucleotide into a copy polynucleotide strand that is complementary to and hybridized with at least a portion of a template polynucleotide strand in a sequencing complex, wherein step (a) is accomplished in the presence of an incorporation mixture that includes less than 1 wt-% sodium ions;
   (b) detecting the identity of the blocked, labeled nucleotide;
   (c) chemically removing a label and blocking moiety from the blocked, labeled nucleotide incorporated into the copy strand;
   (d) repeating steps (a)-(c).
31. A polynucleotide sequencing method comprising:
   (a) incorporating a blocked, labeled nucleotide into a copy polynucleotide strand that is complementary to and hybridized with at least a portion of a template polynucleotide strand in a sequencing complex, wherein step (a) is accomplished in the presence of an incorporation mixture that includes less than 1 wt-% potassium ions;
   (b) detecting the identity of the blocked, labeled nucleotide;
   (c) chemically removing a label and blocking moiety from the blocked, labeled nucleotide incorporated into the copy strand;
   (d) repeating steps (a)-(c).
32. A polynucleotide sequencing method comprising:
   (a) incorporating a blocked, labeled nucleotide into a copy polynucleotide strand that is complementary to and hybridized with at least a portion of a template polynucleotide strand in a sequencing complex, wherein step (a) is accomplished in the presence of an incorporation mixture that includes lithium chloride;
   (b) detecting the identity of the blocked, labeled nucleotide;
   (c) chemically removing a label and blocking moiety from the blocked, labeled nucleotide incorporated into the copy strand;
   (d) repeating steps (a)-(c).

It will also be appreciated from reviewing the present disclosure, that it is contemplated that the one or more aspects or features presented in one of or a group of related clauses may also be included in other clauses or in combination with the one or more aspects or features in other clauses.

All patent and non-patent references and publications cited herein are expressly incorporated herein by reference in their entirety to the extent that they do not conflict with the disclosure presented herein.

A number of embodiments have been described. Nevertheless, it will be understood that various modifications may be made. Accordingly, other embodiments are within the scope of the following claims.

What is claimed is:

1. A polynucleotide sequencing method comprising:
(a) incorporating a blocked, labeled nucleotide into a copy polynucleotide strand that is complementary to and hybridized with at least a portion of a template polynucleotide strand in a sequencing complex;
(b) detecting the identity of the blocked, labeled nucleotide;
(c) chemically removing a label and blocking moiety from the blocked, labeled nucleotide incorporated into the copy strand;
(d) repeating steps (a)-(c),
wherein at least one of steps (a)-(c) are performed in the presence of a single-stranded binding (SSB) protein.

2. The method of claim 1, wherein the method improves secondary structure resolution relative to a sequencing by synthesis process performed in the absence of a SSB protein.

3. The method of claim 2, wherein the method improves G-quadruplex resolution.

4. The method of claim 2, wherein the method comprises:
repeating steps (a)-(c) to sequence at least a portion of a plurality of polynucleotide templates having the same sequence;
wherein completion of sequencing the at least a portion of a plurality of polynucleotides results in a resolution value that is at least 10% greater than a resolution value of the same method completed in the absence of a single-stranded binding protein.

5. The method of claim 2, wherein step (a) is performed in the presence of the single-stranded binding protein.

6. The method of claim 1, wherein the method lowers an error rate of a polynucleotide sequencing by synthesis process relative to a sequencing by synthesis process performed in the absence of a SSB protein.

7. The method of claim 6, wherein the method comprises:
repeating steps (a)-(c) at least 50 times to sequence at least a portion of a plurality of polynucleotide templates having the same sequence;
wherein completion of sequencing the at least a portion of a plurality of polynucleotides results in an error rate that is at least 10% lower than an error rate of the same method completed without a single-stranded binding protein.

8. The method of claim 1, wherein the method increases incorporation kinetics of a polynucleotide sequencing by synthesis process relative to a sequencing by synthesis process performed in the absence of a SSB protein.

9. The method of claim 8, wherein completion of step (a) in the presence of the SSB protein results in an incorporation rate, and wherein the incorporation rate is at least 10% faster than an incorporation rate of performing step (a) in the absence of a single-stranded binding protein.

10. The method of claim 1, wherein:
step (a) further comprises exposing the sequencing complex to an incorporation composition;
step (b) further comprises exposing the sequencing complex to a detection composition;
step (c) further comprises exposing the sequencing complex to a cleavage composition; and
wherein at least one of the incorporation composition, the detection composition, and cleavage composition comprise the single-stranded binding protein.

11. The method of claim 10, wherein the single-stranded binging protein is present in the at least one of the incorporation composition, the detection composition, and cleavage composition at a concentration of 0.01 mg/ml to 2 mg/ml.

12. The method of claim 11, wherein the incorporation composition comprises the single-stranded binding protein.

13. The method of claim 11, wherein the detection composition comprises the single-stranded binding protein.

14. The method of claim 11, wherein the cleavage composition comprises the single-stranded binding protein.

15. The method of claim 1, wherein the method further comprises washing the sequence complex with a wash composition, wherein the wash composition comprises the single-stranded binding protein.

16. The method of any claim 1, wherein at least one of the steps (a)-(c) is performed in the absence of potassium ions.

17. A cartridge for use with a sequencing apparatus, the cartridge comprising:
a first chamber having an incorporation composition for incorporating a blocked, labeled nucleotide into a copy polynucleotide strand complementary to and hybridized with at least a portion of a template polynucleotide strand disposed therein;
a second chamber having a detection composition for detecting the identity of the blocked, labeled nucleotide disposed therein; and
a third chamber having a cleavage composition for chemically removing a label and blocking moiety from the blocked, labeled nucleotide incorporated into the copy strand disposed therein;
wherein at least one of the incorporation composition, the detection composition, and the cleavage composition comprises a single-stranded binding protein.

18. A kit for use with a sequencing apparatus, the kit comprising:
an incorporation composition for incorporating a blocked, labeled nucleotide into a copy polynucleotide strand complementary to and hybridized with at least a portion of a template polynucleotide strand disposed therein;
a detection composition for detecting the identity of the blocked, labeled nucleotide disposed therein; and
a cleavage composition for chemically removing a label and blocking moiety from the blocked, labeled nucleotide incorporated into the copy strand disposed therein;
wherein at least one of the incorporation composition, the detection composition, and the cleavage composition comprises a single-stranded binding protein.

19. The kit of claim 18, further comprising a fourth chamber having a wash composition disposed within, wherein the wash composition comprises a single-stranded binding protein.

20. A polynucleotide sequencing method comprising:
(a) incorporating a blocked, labeled nucleotide into a copy polynucleotide strand that is complementary to and hybridized with at least a portion of a template polynucleotide strand in a sequencing complex, wherein step (a) is accomplished in the presence of an incorporation mixture that includes less than 1 wt-% sodium ions, less than 1 wt-% potassium ions, or lithium chloride;
(b) detecting the identity of the blocked, labeled nucleotide;
(c) chemically removing a label and blocking moiety from the blocked, labeled nucleotide incorporated into the copy strand;
(d) repeating steps (a)-(c).


SEQUENCE LISTING (14 sequences)

SEQ ID NO: 1 - protein, 263 aa, Thermus thermophilus
MARGLNRVFLIGALATRPDMRYTPAGLAILDLTLAGQDLLLSDNGGEREVSWYHRVRLLGRQAEMWGDLLDQGQLVFVEGRLEYRQWEREGERRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDFPPEEELPF

SEQ ID NO: 2 - protein, 283 aa, Thermus thermophilus
MGSSHHHHHHSSGLVPRGSHMARGLNRVFLIGALATRPDMRYTPAGLAILDLTLAGQDLLLSDNGGEREVSWYHRVRLLGRQAEMWGDLLDQGQLVFVEGRLEYRQWEREGERRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDFPPEEELPF

SEQ ID NO: 3 - protein, 266 aa, Thermus thermophilus
GSHMARGLNRVFLIGALATRPDMRYTPAGLAILDLTLAGQDLLLSDNGGEREVSWYHRVRLLGRQAEMWGDLLDQGQLVFVEGRLEYRQWEREGERRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDFPPEEELPF

SEQ ID NO: 4 - protein, 263 aa, Thermus thermophilus
MARGLNRVFLIGALATRPDMRYTPAGLAILDLTLAGQDLLLSDNGGEREVSWYHRVRLLGRQAEMWGDLLDQGQLVFVEGRLEYRQWEREGERRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDPPPEEELPF

SEQ ID NO: 5 - protein, 283 aa, Thermus thermophilus
MGSSHHHHHHSSGLVPRGSHMARGLNRVFLIGALATRPDMRYTPAGLAILDLTLAGQDLLLSDNGGEREVSWYHRVRLLGRQAEMWGDLLDQGQLVFVEGRLEYRQWEREGERRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDPPPEEELPF

SEQ ID NO: 6 - protein, 266 aa, Thermus thermophilus
GSHMARGLNRVFLIGALATRPDMRYTPAGLAILDLTLAGQDLLLSDNGGEREVSWYHRVRLLGRQAEMWGDLLDQGQLVFVEGRLEYRQWEREGERRSELQIRADFLDPLDDRGKERAEDSRGQPRLRAALNQVFLMGNLTRDPELRYTPQGTAVARLGLAVNERRQGAEERTHFVEVQAWRDLAEWAAELRKGDGLFVIGRLVNDSWTSSSGERRFQTRVEALRLERPTRGPAQAGGSRSREVQTGGVDIDEGLEDPPPEEELPF

SEQ ID NO: 7 - protein, 141 aa, Thermotoga maritima
MSFFNKIILIGRLVRDPEERYTLSGTPVTTFTIAVDRVPRKNAPDDAQTTDFFRIVTFGRLAEFARTYLTKGRLVLVEGEMRMRRWETPTGEKRVSPEVVANVVRFMDRKPAETVSETEEELEIPEEDFSSDTFSEDEPPF

SEQ ID NO: 8 - protein, 161 aa, Thermotoga maritima
MGSSHHHHHHSSGLVPRGSHMSFFNKIILIGRLVRDPEERYTLSGTPVTTFTIAVDRVPRKNAPDDAQTTDFFRIVTFGRLAEFARTYLTKGRLVLVEGEMRMRRWETPTGEKRVSPEVVANVVRFMDRKPAETVSETEEELEIPEEDFSSDTFSEDEPPF

SEQ ID NO: 9 - protein, 114 aa, Thermotoga maritima
GSHMSFFNKIILIGRLVRDPEERYTLSGTPVTTFTIAVDRVPRKNAPDDAQTTDFFRIVTFGRLAEFARTYLTKGRLVLVEGEMRMRRWETPTGEKRVSPEVVANVVRFMDRKP

SEQ ID NO: 10 - protein, 148 aa, Thermoproteus tenax
MGEELREEERGEVRSELITKGEKKLVLIRWNTGKTSAGRLFGRYGPGGRPEFFKLLFGAVAGSLREQFGPDGENIFNRIRDSEKFRETSRELFDGLKKWFFEEAVPRYNLERGDIFMISTELVLDPDTGELLWNRDKTQLIYWIRSDR

SEQ ID NO: 11 - protein, 168 aa, Thermoproteus tenax
MGSSHHHHHHSSGLVPRGSHMGEELREEERGEVRSELITKGEKKLVLIRWNTGKTSAGRLFGRYGPGGRPEFFKLLFGAVAGSLREQFGPDGENIFNRIRDSEKFRETSRELFDGLKKWFFEEAVPRYNLERGDIFMISTELVLDPDTGELLWNRDKTQLIYWIRSDR

SEQ ID NO: 12 - protein, 151 aa, Thermoproteus tenax
GSHMGEELREEERGEVRSELITKGEKKLVLIRWNTGKTSAGRLFGRYGPGGRPEFFKLLFGAVAGSLREQFGPDGENIFNRIRDSEKFRETSRELFDGLKKWFFEEAVPRYNLERGDIFMISTELVLDPDTGELLWNRDKTQLIYWIRSDR

SEQ ID NO: 13 - DNA, 24 bp, synthetic construct
  misc_feature 1..24: note = G-quatruplex sequence
  source 1..24: mol_type = other DNA
ttgggggagggggagggggagggg

SEQ ID NO: 14 - DNA, 35 bp, synthetic construct
  misc_feature 1..35: note = dye oligo
  source 1..35: mol_type = other DNA
gtgtagatctcggtggtcgccgtatcattaaaaaa